(19) United States
(12) Patent Application Publication
(10) Pub. No.: US 2021/0363531 A1
(43) Pub. Date: Nov. 25, 2021

(54) TARGETING KIT WITH SPLICE SWITCHING OLIGONUCLEOTIDES TO INDUCE APOPTOSIS OF MAST CELLS

(71) Applicant: North Carolina State University, Raleigh, NC (US)

(72) Inventor: Glenn P. Cruse, Apex, NC (US)

(21) Appl. No.: 17/272,190

(22) PCT Filed: Aug. 27, 2019
(86) PCT No.: PCT/US2019/048400

§ 371 Date: Feb. 26, 2021

Related U.S. Application Data
(60) Provisional application No. 62/723,326, filed on Aug. 27, 2018.

(51) Int. Cl. C12N 15/113; A61P 35/00

(52) U.S. Cl. CPC C12N 15/1135; A61P 35/00; C12N 2320/33; C12N 2310/51; C12N 2310/3233; C12N 2310/11

(57) ABSTRACT

Compositions and methods for modulating Kit biological activities are provided. In some embodiments, the presently disclosed subject matter provides antisense oligomers having 10 to 50 linked nucleotides, wherein the antisense oligomer is targeted to a region of a Kit-encoding pre-mRNA, and further wherein the targeted region includes sequences involved in splicing of the Kit-encoding pre-mRNA. Also provided are expression vectors encoding the antisense oligomers disclosed herein, morpholino oligomer derivatives of the presently disclosed antisense oligomers, pharmaceutical compositions that include the disclosed antisense oligomers, expression vectors, and/or morpholino oligomers, and methods for modulating splicing of a Kit pre-RNA in a cell and/or a tissue, inducing apoptosis in mast cells, and treating diseases, disorders, and/or conditions associated with Kit expression.

### RELATED APPLICATIONS

This application is a U.S. national phase filing based on PCT International Patent Application Serial No. PCT/US2019/048400, filed Aug. 27, 2019, which claims priority to and the benefit of U.S. Provisional Patent Application Ser. No. 62/723,326, filed Aug. 27, 2018; the disclosure of which is incorporated herein by reference in its entirety.

### GRANT STATEMENT

This invention was made with government support under grant number ES025128 awarded by the National Institutes of Health. The government has certain rights in the invention.

### TECHNICAL FIELD

The presently disclosed subject matter relates in some embodiments to targeting Kit gene products, including but not limited to RNA transcripts transcribed form a Kit genomic locus, with splice switching oligonucleotides to induce apoptosis of mast cells. The presently disclosed subject matter relates in some embodiments to applications for selective targeting of mast cells and Kit-related neoplastic diseases.

### INTRODUCTION

c-Kit is a proto-oncogene that is highly conserved between species and encodes the receptor tyrosine kinase, Kit (also known as CD117 and/or the mast/stem cell growth factor receptor (SCFR)). Kit plays a role in the proliferation, survival, and differentiation of bone marrow-derived hematopoietic stem cells, including mast cell (MC) progenitors (reviewed by Pittoni et al., 2011). Expression of Kit in most hematopoietic cells is lost during differentiation, but MCs retain Kit expression throughout their lifespan, and Kit signaling remains essential for MC survival and proliferation (Tsai M et al., 1991; Mekori et al., 1993; Iemura et al., 1994; Galli et al., 1995). The oncogenic potential of c-Kit was initially realized when its viral counterpart, v-Kit, was found to be responsible for the transforming activity of the Hardy-Zuckerman IV feline sarcoma virus (Besmer et al., 1986). Subsequently, activating gain-of-function c-Kit mutations have been associated with the initiation and progression of several human malignancies, with a particularly high occurrence in gastrointestinal stromal tumors (GISTs) and MC proliferative disorders such as mastocytosis and MC leukemia (reviewed in Cruse et al., 2014).

Mastocytosis is a rare and heterogeneous group of neoplastic conditions characterized by aberrant expansion and accumulation of MCs (Valent et al., 2017). The disease can be difficult to treat due to the extensive biological heterogeneity of mastocytosis subtypes, all of which are characterized by the neoplastic growth of MCs but which exhibit distinct clinical manifestations and treatment responses (Valent et al., 2017). Current frontline treatments for mastocytosis and Kit-driven neoplasia include chemotherapy and tyrosine kinase inhibitors (TKIs; Gleixner et al., 2006; Gotlib et al., 2016. However, these treatments are not always effective in advanced disease, in which complete remission or improved prognosis is rare. Moreover, due to the non-specific nature of chemotherapeutics and TKIs, there is a high risk of off-target toxicity (Jensen et al., 2008). Consequently, the development of Kit-specific therapeutic approaches with a better safety profile remains a need in the art.

With a strong connection to cancer development and aberrant MC growth, Kit gene products represent a desirable but challenging therapeutic target. Human c-Kit, located on human chromosome 4q12 (Giebel et al., 1992), contains 21 exons and is susceptible to an array of mutations. The most clinically significant of these are gain-of-function mutations that impart conformational changes in the receptor, and enable tyrosine kinase activity independently from the Kit ligand, stem cell factor (SCF) (reviewed in Cruse et al., 2014; Arock et al., 2015). Wild-type Kit is composed of distinct domains, including an SCF-binding extracellular domain, a transmembrane domain, an autoinhibitory juxtamembrane domain and two intracellular tyrosine kinase domains, which are capable of autophosphorylation (Cruse et al., 2014; Arock et al., 2015). Under normal situations, Kit-SCF interactions are necessary for the survival, proliferation, and differentiation of MCs, but in the presence of activating mutations, SCF-dependence is lost and oncogenic Kit signaling drives neoplastic MC growth.

Although mutations are known occur throughout the Kit gene, many cluster in hotspots, which are associated with different diseases (Cruse et al., 2014). For example, mutations in exon 11, which encodes the juxtamembrane domain of Kit, commonly occur in GISTs (Taniguchi et al., 1999; Miettinen et al., 2002), while the D816V point mutation in exon 17, encoding the second kinase domain, is detected in the majority of patients with systemic mastocytosis (Arock et al., 2015). Identifying patient mutations is important for assessing disease prognosis and determining treatment approaches, because the type of mutation and the occurrence of additional mutations dictate the success of therapies for Kit-associated malignancies. In particular, the missense mutation D816V, found in greater than 80% of adult systemic mastocytosis cases (Arock et al., 2015), causes a conformational change in Kit that renders it resistant to TKIs such as imatinib mesylate (Gleevec; Ma et al., 2002; Pardanani et al., 2003) that target the inactive ATP-binding site conformation of Kit.

Due to the heterogeneity of c-Kit mutations in Kit-associated malignancies and the occurrence of mutations that confer resistance to TKIs disclosed herein in some embodiments are compositions and methods for targeting of Kit expression as an approach to therapy. Since standard siRNA approaches are inefficient at silencing Kit, (Wu et al., 2012; Yang et al., 2015), particularly in vivo, in some embodiments of the presently disclosed subject matter chemically stable antisense oligonucleotides (ASOs) that induce aberrant splicing of pre-mRNA (such as but not limited to exon skipping) are employed to alter the expression of c-Kit gene products. In exemplary embodiments of the presently disclosed subject matter, an exon skipping oligonucleotide (ESO), termed KitStop, targets c-Kit expression by introducing a frameshift into mature c-Kit mRNA transcripts. This results in an immediate STOP codon and loss-of-function. KitStop downregulates both wild-type and mutant Kit expression in MCs, which prevents proliferation and induces rapid cell death in vitro. Administration of KitStop ESO into the peritoneal cavity or skin significantly reduces MC numbers in these tissues in vivo. These data demonstrate that KitStop ESOs can deplete MCs in the animal body, and acts as proof-of-principle for therapeutic utility in Kit-associated malignancies. Given the recent approval of the ESO eteplirsen by the Food and Drug Administration for the treatment of Duchenne muscular dystrophy (DMD; Dowling, 2016; Syed, 2016), the presently disclosed subject matter provides a Kit-targeted therapeutic.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides antisense oligomers comprising 10 to 50 linked nucleotides, wherein the antisense oligomers are targeted to a region of a Kit-encoding pre-mRNA, and further wherein the targeted region comprises sequences involved in splicing of the Kit-encoding pre-mRNA. In some embodiments, hybridization of the antisense oligomer to the Kit-encoding pre-mRNA alters splicing of the pre-mRNA. In some embodiments, hybridization of the antisense oligomer to the Kit-encoding pre-mRNA reduces expression of Kit protein. In some embodiments, the Kit protein for which expression is reduced is a wild type Kit protein or a mutant Kit protein. In some embodiments, the targeted region comprises at least a portion of a polynucleotide sequence selected from the group consisting of an intron sequence, an exon sequence, a sequence comprising an intron/exon junction, a splice donor sequence, a slice acceptor sequence, a splice enhancer sequence, a splice branch point sequence, or a polypyrimidine tract. In some embodiments, the polynucleotide sequence is selected from the group consisting of an exon 4 splice donor sequence. In some embodiments, the Kit-encoding pre-mRNA is transcribed from a c-Kit gene. In some embodiments, the Kit protein is selected from the group consisting of a human Kit protein, a murine Kit protein, a canine Kit protein, a feline Kit protein, and an equine protein. In some embodiments, hybridization of the antisense oligomer to the c-Kit pre-mRNA results in production of a mature c-Kit mRNA molecule that lacks at least a portion of exon 4. In some embodiments, hybridization of the antisense oligomer to the c-Kit pre-mRNA results in production of an mRNA molecule encoding a truncated Kit protein. In some embodiments, the 10 to 50 linked nucleotides comprises a targeting nucleic acid sequence sufficiently complementary to a target nucleic acid sequence in the Kit-encoding pre-mRNA, such that the oligonucleotide specifically hybridizes to the target sequence. In some embodiments, hybridization of the antisense oligomer to the Kit-encoding pre-mRNA alters splicing of the pre-mRNA. In some embodiments, hybridization of the antisense oligomer to the Kit-encoding pre-mRNA reduces expression of Kit protein. In some embodiments, the Kit protein for which expression is reduced is a wild type Kit protein or a mutant Kit protein.

In some embodiments, the targeting sequence comprises at least 6 contiguous nucleobases fully complementary to at least 6 contiguous nucleobases in the target sequence. In some embodiments, the targeting sequence is at least 80% complementary over its entire length to a similarly sized run of contiguous nucleobases in the target sequence. In some embodiments, the target sequence comprises at least a portion of a polynucleotide sequence selected from the group consisting of an intron sequence, an exon sequence, a sequence comprising an intron/exon junction, a splice donor sequence, a slice acceptor sequence, a splice enhancer sequence, a splice branch point sequence, or a polypyrimidine tract. In some embodiments, the polynucleotide sequence is associated with a Kit exon selected from the group consisting of Kit exons 2-20, such as but not limited to an exon 4 splice donor sequence. In some embodiments, the Kit-encoding pre-mRNA is transcribed from a c-Kit gene. In some embodiments, the Kit protein is selected from the group consisting of a human Kit protein, a murine Kit protein, a canine Kit protein, a feline Kit protein, and an equine protein.

In some embodiments, the target sequence comprises at least a portion of a polynucleotide sequence selected from the group consisting of SEQ ID NOs: 18-22, optionally SEQ ID NOs: 23-60. In some embodiments, the portion is at least 10 contiguous nucleotides. In some embodiments, the target sequence comprises a sequence at least 90% identical to a subsequence of SEQ ID NOs: 18-22, optionally SEQ ID NOs: 23-60. In some embodiments, the target sequence comprises a sequence selected from the group consisting of the reverse complement of SEQ ID NOs: 1 and 2 and SEQ ID NOs: 23-60. In some embodiments, the targeting sequence comprises at least 10 contiguous nucleobases identical in sequence to at least 10 contiguous nucleobases in a sequence selected from the group consisting of SEQ ID NOs: 1, 2, and the reverse complement of one of SEQ ID NOs: 23-60. In some embodiments, the targeting sequence comprises a sequence at least 80% complimentary to the reverse complement of at least a portion of a subsequence of SEQ ID NOs: 18-22, optionally SEQ ID NOs: 23-60. In some embodiments, the targeting sequence is at least 80% identical over the full length of a sequence selected from the group consisting of SEQ ID NOs: 1, 2, and the reverse complement of one of SEQ ID NOs: 23-60. In some embodiments, the targeting sequence is selected from the group consisting of SEQ ID NOs: 1, 2, and the reverse complement of one of SEQ ID NOs: 23-60.

In some embodiments, the c-Kit transcript comprises any of SEQ ID NOs: 8, 10, 12, 14, and 16, or an open reading frame present therein.

In some embodiments, the antisense oligomer is an antisense RNA molecule. In some embodiments, the antisense RNA molecule comprises a modification selected from the group consisting of a nucleotide modification, an internucleotide modification, a sugar modification, a sugar-internucleotide linkage modification, and combinations thereof. In some embodiments, the antisense oligomer is a morpholino oligomer.

In some embodiments, the presently disclosed subject matter also provides expression vectors encoding an antisense oligomer as described herein.

In some embodiments, the presently disclosed subject matter also provides pharmaceutical compositions comprising the antisense oligomers described herein, the expression vectors described herein, and/or the morpholino oligomers described herein.

In some embodiments, the presently disclosed subject matter also provides methods for modulating splicing of Kit-encoding pre-RNAs in cells and/or tissues. In some embodiments, the methods comprise contacting the cells and/or the tissues with an antisense oligomer as described herein, an expression vector as described herein, and/or a morpholino oligomer as described herein.

In some embodiments, the presently disclosed subject matter also provides methods for inducing apoptosis in mast cells. In some embodiments, the presently disclosed methods comprise contacting the mast cells with an antisense oligomer as described herein, an expression vector as described herein, and/or a morpholino oligomer as described herein.

In some embodiments of the presently disclosed methods, the methods are performed in an individual.

In some embodiments, the presently disclosed subject matter also provides methods for treating diseases, disorders, and/or conditions associated with Kit expression in individuals. In some embodiments, the methods comprise administering to an individual an antisense oligomer as described herein, an expression vector as described herein, and/or a morpholino oligomer as described herein. In some embodiments, a disease, disorder, and/or condition associated with Kit expression is a cancer or mastocytosis. In some embodiments, the cancer is a gastrointestinal stromal tumor or leukemia. In some embodiments, the individual is an animal, optionally a mammal. In some embodiments, the individual is a human, a mouse, a dog, a cat, or a horse.

Thus, it is an object of the presently disclosed subject matter to provide for targeting Kit gene products with splice switching oligonucleotides to induce apoptosis of mast cells and for selective targeting of mast cells and Kit-related neoplastic diseases

An object of the presently disclosed subject matter having been stated hereinabove, and which is achieved in whole or in part by the compositions and methods disclosed herein, other objects will become evident as the description proceeds when taken in connection with the accompanying Figures as best described herein below.

### BRIEF DESCRIPTION OF THE FIGURES

The accompanying drawings, which are incorporated herein by reference and constitute a part of this specification, illustrate several representative embodiments of the presently disclosed subject matter and together with the description illustrate the disclosed compositions and methods.

FIGS. 1A-1C: ESO-mediated alternative splicing of exon 4 in c-Kit pre-mRNA. (FIG. 1A) KitStop ESO was designed to target the donor splice site of exon 4, which led to exclusion of exon 4 by the spliceosome. This is predicted to introduce a premature stop codon and result in a truncated mRNA transcript, even in the presence of G560V and D816V activating mutations, which are located downstream of the target site of to KitStop. Boxes represent exons; thick black bar represents introns. FL-c-Kit=full-length c-Kit, t-c-Kit=truncated c-Kit. (FIG. 1B) Gel electrophoresis data demonstrating splice-switching of wild-type and mutant c-Kit by KitStop ESO in comparison to standard control ASO (Stndcon) in HMC-1.2 (FIG. 1B) and LAD2 cells (FIG. 1C), respectively, as assessed by analysis of total RNA by RT-PCR. Black arrow=full-length c-Kit, gray arrow=alternatively spliced c-Kit.

FIGS. 2A-2F: Transfection of KitStop ESO leads to loss of wild-type Kit expression. Following transfection of LAD2 cells with 10 μM standard control ASO (Stndcon) or KitStop ESO, Kit expression was assessed by flow cytometry. (FIG. 2A) Flow cytometry histograms of surface (FIG. 2A) and total (FIG. 2B) wild-type Kit expression at Day 2 (top panels) and Day 7 (bottom panels) following transfection with KitStop ESO. Mean flow cytometry data for surface (FIG. 2C) and total (FIG. 2D) Kit expression calculated from the geometric MFI and expressed as a percentage of Stndcon. Data are the mean±SEM from 3 independent experiments. ***p<0.001, ****p<0.0001, ANOVA with Sidak's post-test. Representative flow cytometry gating strategies and plots of LAD2 cells labeled with anti-human CD117 antibodies are provided in FIGS. 2E and 2F. Following transfection of LAD2 cells with 10 μM KitStop ESO, LAD2 cells were assessed by flow cytometry for Kit expression. Representative density plots and gates of live (FIG. 2E), and fixed and permeabilized (FIG. 2F) LAD2 cells labeled with APC-conjugated anti-human CD117 antibody. Stndcon=standard control ASO. I: isotype; S: Stndcon; K: KitStop.

FIGS. 3A-3F: Transfection of KitStop ESO leads to loss of mutant Kit expression. Following transfection of HMC-1.2 cells with 10 μM standard control ASO (Stndcon) or KitStop ESO, HMC-1.2 cells were assessed by flow cytometry for Kit expression. (FIG. 3A) Flow cytometry histograms of total Kit expression at 24 (top panel) 48 (middle panel) and 72 h (bottom panel) following transfection with KitStop ESO. (FIG. 3B) Mean flow cytometry data for total Kit expression calculated from the geometric mean fluorescence intensity (MFI) (FIG. 3B) and expressed as a percentage of standard control (Stndcon) ASO (FIG. 3C). Data are the mean±SEM from 3 independent experiments. *p<0.05, **p<0.01, ***p<0.001, ANOVA with Sidak's post-test. Representative flow cytometry gating strategies and plots of HMC-1.2 cells labeled with anti-human CD117 antibodies. Representative density plots and gates of fixed and permeabilized HMC-1.2 cells transfected with 10 μM KitStop ESO and labeled with APC-ft) conjugated anti-human CD117 antibody after 24 hours (FIG. 3D), 48 hours (FIG. 3E), and 72 hours (FIG. 3F). KitStop treated cells have reduced Kit expression and reduced forward scatter, indicating cell shrinkage. Stndcon=standard control ASO. I: isotype; S: Stndcon; K: KitStop.

FIGS. 4A-4C. KitStop reduces constitutive KIT signaling in HMC-1.2 cells. (FIG. 4A) Immunoblots of constitutive phosphorylation of KIT and ERK in HMC-1.2 cells after 24 h of KitStop treatment from three independent experiments shown from left to right. (FIGS. 4B and 4C) Combined phosphorylation data for pKIT (FIG. 4B) and pERK (FIG. 4C) after correction against β-actin or total ERK, respectively, and normalized to phosphorylation in control cells. Data are the mean±SEM from 3 independent experiments. *p<0.05 using a Student's paired t-test.

FIGS. 5A-5G: Transfection of KitStop ESO increases HMC-1.2 cell apoptosis. Following transfection of HMC-1.2 cells with 10 μM standard control ASO (Stndcon) or KitStop ESO, HMC-1.2 cells were assessed by flow cytometry for apoptosis by staining with Annexin V-FITC. (FIG. 5A) Histograms showing the shift in Annexin V positive staining of KitStop-transfected cells in comparison to Stndcon and unstained cells. (FIG. 5B) Combined data from flow cytometry for Annexin V expressed as the geometric MFI. (FIG. 5C) Percentage of HMC-1.2 cells within non-apoptotic (FIG. 5C), early apoptotic (FIG. 5D) and late apoptotic (FIG. 5E) gates at each time point. Data are the mean±SEM from 3 independent experiments. *p<0.05, **p<0.01, ***p<0.001, ANOVA with Sidak's post-test. Representative flow cytometry gating strategies and plots of HMC-1.2 cells stained with Annexin V-FITC. (FIG. 5F) Gating strategy and representative density plots of live, untreated HMC-1.2 cells, either unstained or stained with Annexin V-FITC. (FIG. 5G) Flow cytometry density plots of Annexin V staining intensity of HMC-1.2 cells at each time point after transfection with standard control ASO (Stndcon) or KitStop ESO. Apoptotic cells are smaller when measured by forward scatter (X axes) and become annexin V positive (Y axes). U: unlabeled; S: Stndcon; K: KitStop.

FIGS. 6A-6J: Transfection of KitStop ESO decreases HMC-1.2 cell viability. Following transfection of HMC-1.2 cells with 10 μM standard control ASO (Stndcon) or KitStop ESO, HMC-1.2 cell viability was assessed by flow cytometry by staining with propidium iodide (PI). Percentage of PI negative and positive cells at 24 h (FIG. 6A), 48 h (FIG. 6B), and 72 h (FIG. 6C). (FIG. 6D) Flow cytometry histograms at 24 (left panel), 48 (middle panel) and 72 h (right panel) of HMC-1.2 cells stained with LIVE/DEAD Green Dead Cell stain. (FIG. 6E) Combined geometric MFI of LIVE/DEAD staining in HMC-1.2 cells at each time point. Data are the mean±SEM from 3 independent experiments. *p<0.05, **p<0.01, ***p<0.001, ANOVA with Sidak's post-test. Representative flow cytometry gating strategies and plots of HMC-1.2 cells stained with propidium iodide. (FIG. 6F) Representative density plots of HMC-1.2 cells stained with propidium iodide (PI). Flow cytometry density plots of PI staining intensity of HMC-1.2 cells at each time point after transfection with standard control ASO (Stndcon) (FIG. 6G) or KitStop ESO (FIG. 6H). Flow cytometry gating strategies for HMC-1.2 cells with LIVE/DEAD stain. (FIG. 6I) Representative density plots demonstrating (FIG. 6I) gating of live HMC-1.2 cells and single cell populations and (FIG. 6J) gating of untreated cells following application of LIVE/DEAD Green Dead Cell stain. U: unlabeled; S: Stndcon; K: KitStop.

FIGS. 7A-7E: Transfection of KitStop ESO decreases HMC-1.2 cell proliferation. Following transfection of HMC-1.2 cells with 10 μM standard control ASO (Stndcon) or KitStop ESO, HMC-1.2 cell proliferation was assessed by cell counts and flow cytometry. (FIG. 7A) Total number of viable HMC-1.2 cells cultured under normal conditions assessed by Trypan blue counts. (FIG. 7B) Representative density plots of HMC-1.2 cells loaded with CellTrace prior to transfection with Stndcon ASO (top panels) or KitStop ESO (bottom panels) and stained with LIVE/DEAD Green Dead Cell stain at 24 (left panels) 48 (middle panels) and 72 h (right panels). Cells transfected with Stndcon demonstrated a loss of CellTrace fluorescence intensity due to dye dilution between daughter cells, indicating cell proliferation. In contrast, KitStop-transfected cells retained CellTrace fluorescence, except for a small population at 72 h, and exhibited increased LIVE/DEAD staining intensity. (FIG. 7C) Percentage of total cells in bottom left quadrants (CellTrace^{low}; LIVE/DEAD^{negative}) corresponding to proliferating cells. (FIG. 7D) Percentage of total cells in the top and bottom right quadrants corresponding to non-proliferating cells (CellTrace^{high}). (FIG. 7E) Percentage of total cells in top left and right quadrants (LIVE/DEAD⁺), corresponding to non-viable cells. Data are the mean±SEM from 3 independent experiments. *p<0.05, ***p<0.001, ****p<0.0001, ANOVA with Sidak's post-test.

FIGS. 8A-8H: KitStop ESO reduces wild-type Kit expression in mouse mast cells in vitro and in vivo. (FIG. 8A) RT-PCR demonstrating splice-switching of wild-type c-Kit in bone marrow-derived mast cells (BMMCs) by mouse KitStop ESO in comparison to standard control ASO (Stndcon). Black arrow=full-length c-Kit, gray arrow=alternatively spliced c-Kit. (FIG. 8B) Flow cytometry histogram of surface wild-type Kit expression in BMMCs following transfection with KitStop ESO. Data are representative of 3 independent experiments on separate mice. (FIG. 8C) Mean flow cytometry data for surface Kit expression calculated from the geometric MFI and expressed as a percentage of Stndcon ASO. Data are the mean±SEM from 3 independent experiments on separate mice. *p<0.01, paired t-test. (FIG. 8D) Timeline for intraperitoneal delivery of KitStop Vivo Morpholinos. (FIG. 8E) RT-PCR for c-Kit expression, with evidence of splice switching, in cells collected by peritoneal lavage. Black arrow=full-length c-Kit, gray arrow=alternatively spliced c-Kit. (FIG. 8F) Representative flow cytometry density plots of peritoneal cells harvested from mice treated with Stndcon ASO (FIG. 8F), or KitStop ESO (FIG. 8G) Vivo Morpholinos. (FIG. 8H) Percentage of mast cells, identified as Kit and FcεRI double positive cells (top right quadrant in FIG. 8F and FIG. 8G), harvested by peritoneal lavage following treatment with Stndcon ASO or KitStop ESO Vivo Morpholinos. Each datapoint represent a different mouse and data are combined from two independent experiments (p value from unpaired t-test). I: isotype; S: Stndcon; K: KitStop.

FIGS. 9A-9E: KitStop ESO reduces mouse skin mast cell numbers in vivo. (FIG. 9A) Timeline for skin injection of KitStop Vivo Morpholinos. (FIG. 9B) In representative sections from standard control ASO (Stndcon) and KitStop ESO treatment groups, mast cells were easily identified due to the presence of positive-staining metachromatic granules, which give a deep violet hue (see red arrow heads). (FIG. 9C) Skin histology with hematoxylin and eosin staining from Stndcon ASO and KitStop ESO treated mice displayed normal morphology with no evidence of pathology. (FIG. 9D) Plot of dermal mast cell number per mm² from toluidine blue stained skin sections taken from Stndcon ASO or KitStop ESO treated skin. (FIG. 9E) Plot of mast cell number per mm² from the full thickness skin sections, including dermis and panniculus adiposus surrounding adnexal structures. Each dot represents the average mast cell number per entire specimen. P values are from an unpaired t-test.

FIGS. 10A-10G. Systemic delivery of KitStop ESO inhibits tumor growth in a humanized xenograft mast cell neoplasia model. (FIG. 10A) Schematic representation of the protocol for the xenograft model used. (FIG. 10B) Measurements of tumor volume over time during treatment. Arrows indicate days that KitStop or vehicle control was administered. (FIG. 10C) Weight of excised tumors after mice were euthanized on day 14. (FIG. 10D) Photograph of tumors after mice were euthanized at day 14. Measurements are in centimeters. (FIG. 10E) Spleen weight at the conclusion of the experiment. (FIG. 10F) Liver weight at the conclusion of the experiment. (FIG. 10G) Mice weight was monitored over the course of the experiment. No significant differences were observed over the course of the experiment. Data are the mean±SEM from 5 mice per group. *p<0.05, **p<0.01, ***p<0.001. ANOVA with Dunnett's post-test (FIG. 10B) or unpaired t-test (FIGS. 10C and 10E).

### BRIEF DESCRIPTION OF THE SEQUENCE LISTING

SEQ ID NO: 1 is the nucleotide sequence of a KitStop ESO designed to target exon 4 of an exemplary human c-Kit gene product (Accession No. NM 000222.2 of the GENBANK® biosequence database) wherein a region within the splicing donor site was targeted. SEQ ID NO: 1 is designed to hybridize specifically to the 3′ six nucleotides of exon 4 of the human KIT gene product represented by Accession No. NM_000222.2 of the GENBANK® biosequence database and the 19 nucleotides in the downstream intron. As such, SEQ ID NO: 1 corresponds to the reverse complement of nucleotides 41,835-41,859 of SEQ ID NO: 18.

SEQ ID NO: 2 is the nucleotide sequence of an ESO for an exemplary murine c-kit gene product (Accession No. NM_001122733.1 of the GENBANK® biosequence database). SEQ ID NO: 2 is designed to hybridize specifically to the 3 six nucleotides of exon 4 of the Mus musculus Kit gene product represented by Accession No. NM_001122733.1 of the GENBANK® biosequence database and the 19 nucleotides in the downstream intron. As such, SEQ ID NO: 2 corresponds to the reverse complement of nucleotides 35,953-35,977 of SEQ ID NO: 19.

SEQ ID NO: 3 is the nucleotide sequence of a standard control ASO has identical chemistry to the KitStop ESO but it does not induce exon skipping of any known gene.

SEQ ID NOs: 4 and 5 are the nucleotide sequences of forward and reverse primers that can be used together to amplify exon 4 of human c-KIT mRNA.

SEQ ID NOs: 6 and 7 are the nucleotide sequences of forward and reverse primers that can be used together to amplify exon 4 of mouse c-kit mRNA.

SEQ ID NOs: 8 and 9 are nucleotide and amino acid sequences, respectively, of exemplary human c-KIT gene products. The nucleotide sequence corresponds to Accession No. NM 000222.2 in the GENBANK® biosequence database, and the amino acid sequence corresponds to Accession No. NP_000213.1 in the GENBANK® biosequence database. The transmembrane domain of the exemplary human c-KIT gene product corresponds to amino acids 525-545 of SEQ ID NO: 9 and is encoded within exon 10. Any truncation prior to this should result in some embodiments in a defective protein and in some embodiments, should result is a failure of the protein to be produced and/or to localize to the cell membrane.

SEQ ID NOs: 10 and 11 are nucleotide and amino acid sequences, respectively, of exemplary murine c-kit gene products. The nucleotide sequence corresponds to Accession No. NM_001122733.1 in the GENBANK® biosequence database, and the amino acid sequence corresponds to Accession No. NP_001116205.1 in the GENBANK® biosequence database.

SEQ ID NOs: 12 and 13 are nucleotide and amino acid sequences, respectively, of exemplary feline c-kit gene products. The nucleotide sequence corresponds to Accession No. NM_001009837.3 in the GENBANK® biosequence database, and the amino acid sequence corresponds to Accession No. NP_001009837.3 in the GENBANK® biosequence database.

SEQ ID NOs: 14 and 15 are nucleotide and amino acid sequences, respectively, of exemplary canine c-kit gene products. The nucleotide sequence corresponds to Accession No. NM_001003181.1 in the GENBANK® biosequence database, and the amino acid sequence corresponds to Accession No. NP_001003181.1 in the GENBANK® biosequence database.

SEQ ID NOs: 16 and 17 are nucleotide and amino acid sequences, respectively, of exemplary equine c-kit gene products. The nucleotide sequence corresponds to Accession No. NM_001163866.2 in the GENBANK® biosequence database, and the amino acid sequence corresponds to Accession No. NP_001157338.2 in the GENBANK® biosequence database.

SEQ ID NO: 18 is an exemplary genomic sequence of the human c-KIT locus. It corresponds to nucleotides 54,657,928-54,740,715 of Accession No. NC 000004.12 in the GENBANK® biosequence database.

SEQ ID NO: 19 is an exemplary genomic sequence of the murine c-kit locus. It corresponds to nucleotides 75,574,987-75,656,745 of Accession No. NC 000071.6 in the GENBANK® biosequence database.

SEQ ID NO: 20 is an exemplary genomic sequence of the feline c-kit locus. It corresponds to the reverse complement of nucleotides 163,952,966-164,039,337 of Accession No. NC 018726.3 in the GENBANK® biosequence database.

SEQ ID NO: 21 is an exemplary genomic sequence of the canine c-kit locus. It corresponds to nucleotides 47,108,504-47,190,020 of Accession No. NC 006595.3 in the GENBANK® biosequence database.

SEQ ID NO: 22 is an exemplary genomic sequence of the equine c-kit locus. It corresponds to the reverse complement of nucleotides 79,538,697-79,618,653 of Accession No. NC 009146.3 in the GENBANK® biosequence database.

SEQ ID NOs: 23-60 are target nucleotide sequences for additional exemplary ESOs that are designed to target one of exons 2-20 of an exemplary human c-KIT gene product (Accession No. NM_000222.2 of the GENBANK® biosequence database) wherein a region that includes a splice donor site or a splice acceptor site can be targeted. See Table 3. Particularly, SEQ ID NOs: 23 and 24 can be employed to skip exon 2, resulting in an in frame deleted nucleotide sequence encoding SEQ ID NO: 61; SEQ ID NOs: 25 and 26 can be employed to skip exon 3, resulting in an in frame deleted nucleotide sequence encoding SEQ ID NO: 62; SEQ ID NOs: 27 and 28 can be employed to skip exon 4, resulting in a frame shifted nucleotide sequence encoding SEQ ID NO: 63; SEQ ID NOs: 29 and 30 can be employed to skip exon 5, resulting in a frame shifted nucleotide sequence encoding SEQ ID NO: 64; SEQ ID NOs: 31 and 32 can be employed to skip exon 6, resulting in a frame shifted nucleotide sequence encoding SEQ ID NO: 65; SEQ ID NOs: 33 and 34 can be employed to skip exon 7, resulting in a frame shifted nucleotide sequence encoding SEQ ID NO: 66; SEQ ID NOs: 35 and 36 can be employed to skip exon 8, resulting in a frame shifted nucleotide sequence encoding SEQ ID NO: 67; SEQ ID NOs: 37 and 38 can be employed to skip exon 9, resulting in a frame shifted nucleotide sequence encoding SEQ ID NO: 68; SEQ ID NOs: 39 and 40 can be employed to skip exon 10, resulting in a frame shifted nucleotide sequence encoding SEQ ID NO: 69, which since exon 10 encodes the transmembrane domain can in some embodiments create a soluble receptor that is incapable of signaling; SEQ ID NOs: 41 and 42 can be employed to skip exon 11, resulting in a frame shifted nucleotide sequence encoding SEQ ID NO: 70, which since exon 11 encodes amino acids just C-terminal to the transmembrane domain, can in some embodiments create a kinase dead, dominant negative protein; SEQ ID NOs: 43 and 44 can be employed to skip exon 12, resulting in an in frame deleted nucleotide sequence encoding SEQ ID NO: 71; SEQ ID NOs: 45 and 46 can be employed to skip exon 13, resulting in an in frame deleted nucleotide sequence encoding SEQ ID NO: 72; SEQ ID NOs: 47 and 48 can be employed to skip exon 14, resulting in a frame shifted nucleotide sequence encoding SEQ ID NO: 73; SEQ ID NOs: 49 and 50 can be employed to skip exon 15, resulting in a frame shifted nucleotide sequence encoding SEQ ID NO: 74; SEQ ID NOs: 51 and 52 can be employed to skip exon 16, resulting in a frame shifted nucleotide sequence encoding SEQ ID NO: 75; SEQ ID NOs: 53 and 54 can be employed to skip exon 17, resulting in an in frame deleted nucleotide sequence encoding SEQ ID NO: 76; SEQ ID NOs: 55 and 56 can be employed to skip exon 18, resulting in a frame shifted nucleotide sequence encoding SEQ ID NO: 77; SEQ ID NOs: 57 and 58 can be employed to skip exon 19, resulting in a frame shifted nucleotide sequence encoding SEQ ID NO: 78; and SEQ ID NOs: 59 and 60 can be employed to skip exon 20, resulting in a frame shifted nucleotide sequence encoding SEQ ID NO: 79.

SEQ ID NOs: 61-79 are the amino acid sequences of human KIT polypeptides that would result from exon skipping using the ESOs of SEQ ID NOs: 23-60. Particularly, SEQ ID NO: 61 is the predicted amino acid sequence of an exon 2 skipped protein, SEQ ID NO: 62 is the predicted amino acid sequence of an exon 3 skipped protein, SEQ ID NO: 63 is the predicted amino acid sequence of a severely truncated exon 4 skipped protein, SEQ ID NO: 64 is the predicted amino acid sequence of a severely truncated exon 5 skipped protein, SEQ ID NO: 65 is the predicted amino acid sequence of a severely truncated exon 6 skipped protein, SEQ ID NO: 66 is the predicted amino acid sequence of a severely truncated exon 7 skipped protein, SEQ ID NO: 67 is the predicted amino acid sequence of a severely truncated exon 8 skipped protein, SEQ ID NO: 68 is the predicted amino acid sequence of a severely truncated exon 9 skipped protein, SEQ ID NO: 69 is the predicted amino acid sequence of a severely truncated exon 10 skipped protein, SEQ ID NO: 70 is the predicted amino acid sequence of a severely truncated exon 11 skipped protein, SEQ ID NO: 71 is the predicted amino acid sequence of an exon 12 skipped protein, SEQ ID NO: 72 is the predicted amino acid sequence of an exon 13 skipped protein, SEQ ID NO: 73 is the predicted amino acid sequence of an exon 14 skipped protein, SEQ ID NO: 74 is the predicted amino acid sequence of an exon 15 skipped protein, SEQ ID NO: 75 is the predicted amino acid sequence of an exon 16 skipped protein, SEQ ID NO: 76 is the predicted amino acid sequence of an exon 17 skipped protein, SEQ ID NO: 77 is the predicted amino acid sequence of an exon 18 skipped protein, SEQ ID NO: 78 is the predicted amino acid sequence of an exon 19 skipped protein, and SEQ ID NO: 79 is the predicted amino acid sequence of an exon 20 skipped protein.

### DETAILED DESCRIPTION

Activating mutations in the proto-oncogene c-kit are associated with malignancies including systemic mastocytosis and mast cell (MC) leukemia. c-kit encodes Kit (CD117; mast/stem cell growth factor receptor (SCFR)), a receptor tyrosine kinase that plays a role in MC proliferation and is required for MC survival. Therefore, therapies blocking Kit signaling are a leading strategy to treat MC proliferative disorders.

However, despite some success, current therapies have off-target effects and, in some patients, complete remission or improved survival time cannot be achieved. This is due, in part, to c-kit mutations that confer resistance to tyrosine kinase inhibitors. Disclosed herein in some embodiments of the presently disclosed subject matter are innovative approaches to specifically target Kit expression with exon skipping oligonucleotides (ESOs) that introduce frameshifts into mature mRNA of either wild-type or mutant c-kit resulting in downregulation of Kit expression, inhibition of neoplastic MC proliferation, and induction of MC apoptosis. In addition, ESO administration depletes tissue MCs in vivo demonstrating therapeutic potential.

The therapeutic potential of ESOs has been significantly enhanced by chemical modifications that improve stability and bioavailability. Currently, ESOs have clear clinical applications in genetic diseases resulting from loss-of-function frameshift mutations, such as Duchenne muscular dystrophy, where ESOs restore the reading frame of the mature mRNA transcript, resulting in translation of an alternatively spliced, but partially functional protein. In some embodiments, the presently disclosed subject matter provides an ESO that achieves the opposite, and disrupts the open reading frame of an oncogenic transcript resulting in a loss-of-function frameshift in mature mRNA. In some embodiments of the presently disclosed subject matter, targeting Kit resulted in rapid and efficient MC death and reduction of MC numbers in vivo, demonstrating a Kit-targeted therapeutic to address the unmet clinical need for Kit-associated malignancies.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

While the following terms are believed to be well understood by one of ordinary skill in the art, the following definitions are set forth to facilitate explanation of the presently disclosed subject matter.

As used herein and in the appended claims, the singular forms “a”, “an”, and “the” include plural referents unless the context clearly dictates otherwise. For example, a nucleic acid molecule refers to one or more nucleic acid molecules. As such, the terms “a”, “an”, “one or more”, and “at least one” can be used interchangeably. Similarly, the terms “comprising”, “including”, and “having” can be used interchangeably. It is further noted that the claims may be drafted to exclude any optional element. As such, this statement is intended to serve as antecedent basis for use of such exclusive terminology as “solely”, “only”, and the like, in connection with the recitation of claim elements, or use of a “negative” limitation.

Ranges can be expressed herein as from “about” one particular value, and/or to “about” another particular value. When such a range is expressed, some embodiments includes from the one particular value and/or to the other particular value. Similarly, when values are expressed as approximations, by use of the antecedent “about”, it will be understood that the particular value forms an embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” are also disclosed. It is also understood that the throughout the application, data are provided in a number of different formats, and that these data represent in some embodiments endpoints and starting points and in some embodiments ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “and/or”, when used in the context of a list of entities, refers to the entities being present singly or in combination.

The terms “optional” and “optionally” as used herein indicate that the subsequently described event, circumstance, element, and/or method step may or may not occur and/or be present, and that the description includes instances where said event, circumstance, element, or method step occurs and/or is present as well as instances where it does not.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### II. General Considerations

Human malignancies associated with activating c-KIT mutations include mast cell proliferative disorders, gastrointestinal stromal tumors (GISTs), and less commonly, melanoma and acute myeloid leukemia (AML). Increased expression of normal c-KIT may also contribute to tumorigenesis in solid lung cancers from small lung cells that do not normally express KIT and are exposed to environments rich in SCF. GISTs are believed to derive from interstitial cells of Cajal and in up to 80% of sporadic GISTs, at least 17 different activating mutations involving exons 8, 11, 13, or 17 of c-Kit have been reported. Similarly, approximately 90% of adults with diseases of abnormal mast cell proliferation (mastocytosis) have at least a point mutation consisting of a substitution of aspartic acid to valine in the catalytic domain of c-KIT (D816V), rendering it constitutively active, and/or other mutations in c-KIT.

Therefore, therapies blocking KIT activity are the main approaches to treat these patient populations and they have been somewhat successful in the treatment of some of these malignancies alone or in combination, but not in others where complete remissions or improved survival time is rare. The success of therapy is linked to the type of mutation and/or the presence of additional mutations in c-KIT or other proto-oncogenes. Pharmacological targeting of KIT catalytic activity has been a major strategy for blocking KIT-mediated responses. A limiting factor in the treatment of KIT-related malignancies has to been that inhibitors targeting the inactive ATP-binding site conformation of KIT, such as imatinib mesylate (imatinib, STI571, Gleevec, or Glivec) and its derivatives (nilotinib (AMN107) and PD180970), are unsuccessful in inhibiting the active, extended conformation of KIT. Therefore, mutations that stabilize the active conformation (mostly kinase domain mutations, including the common D816V mutation), as those found in most aggressive mastocytosis patients, are resistant to this drug. An additional drawback of imatinib has been the incidence in some patients of secondary resistance to the drug, apparently due in part to the acquisition (or enrichment) of other mutations in the kinase domain of the receptor that are insensitive to imatinib. Therefore, patients may initially respond to imatinib that eliminates the imatinib sensitive cells, but may have a reoccurrence of imatinib-resistant clonal expansions.

KIT-associated neoplastic diseases are often treated by drugs that are in no way specific and thus have many off-target effects. Imatinib is the drug of choice because it is more specific to KIT signaling than other drugs as it fits into the kinase pocket of KIT. However, many patients, particularly those with the most severe disease, are resistant to imatinib and more general inhibitors are used that have wide-ranging and often severe side effects. In some embodiments, the presently disclosed subject matter is targeted to KIT and thus off-target effects are minimal compared to existing kinase inhibitors that are commonly used.

Other compounds, including dasatinib (BMS-354825), have been found to target the catalytic activity associated with D816V and other KD mutations. Dasatinib was reported to inhibit Kit autophosphorylation and the growth of both human mast cell line (HMC)-1.1 and HMC-1.2 human mast cell lines, which express the V560G mutation or the V560G and D816V mutations, respectively. The HMC1.2 mast cell line therefore harbors the mutation in the kinase domain that confers resistance to imatinib and other inhibitors. Dasatinib and derivatives, however, have wider specificity and affect multiple kinases such as Src Kinases, Tec kinases, Bruton's tyrosine kinase (Btk), mitogen-activated protein kinases and AKT as well as other receptor kinases that are important for growth and other functions in mast cells and other cell types. Thus, while being potentially more effective by acting on various pathways, their distinct side effects should also be taken into consideration depending on the patient.

The presently disclosed subject matter provides a treatment for Kit-mediated responses that eliminates expression of Kit in mast cells, leading to loss of proliferation, induction of apoptosis, and rapid cell death of transformed HMC1.2 mast cell leukemia cells that harbor the V560G and D816V mutations. These cells are used to test new drugs for mastocytosis and they are resistant to many kinase inhibitors due to the conformation of the constitutively active Kit that they express. The presently disclosed subject matter kills these cells within hours and it is predicted that it will effectively kill cells that harbor any activating Kit mutation. In some embodiments, the presently disclosed subject matter utilizes chemically stable splice switching oligonucleotides to alter the splicing of Kit pre-mRNA that induces exon skipping of exon 4 (a very early exon in the mRNA) to introduce a frame-shift in the open reading frame that results in an immediate STOP codon and thus eliminates receptor expression.

The presently disclosed subject matter can be utilized in Kit-associated cancer and neoplastic diseases, and also has applications in mast cell-associated diseases since administering the drug specifically depletes mast cells in tissue. Depleting mast cells can relieve allergy-like symptoms that are driven by mast cells that may be hyperresponsive. Also, mast cell numbers are increased in tissues of allergic patients and administration can deplete these cells. Kit expression outside of the bone marrow is restricted to a few cell types and thus, the presently disclosed subject matter is targeted. Mast cell depletion in tissue can also be utilized as a research tool where studies of mast cell function are enabled if comparisons between tissue containing mast cells can be compared to tissue that is mast cell deficient. Kit deficient mice have been used for these studies, but these mice have other immune issues associated with functions for Kit in the bone marrow. Finally, the presently disclosed subject matter provides for the treatment of mast cell tumors, which are the most common tumor in dogs (and to some degree cats also). The presently disclosed subject matter can be used to kill mast cell tumor cells, given its high potency in human mast cell leukemia cells.

### III. Oligonucleotides and Related Methods

Antisense technology has been demonstrated to be an effective method of modifying the expression levels of gene products (see for example, U.S. Pat. Nos. 8,765,703, 8,946,183, and U.S. Patent Publication No. 2015/0376615, which are incorporated herein by reference in their entirety). Antisense technology works by interfering with known steps in the normal processing of mRNA. Briefly, RNA molecules are transcribed from genomic DNA in the nucleus of the cell. These newly synthesized mRNA molecules, called primary mRNA or pre-mRNA, must be processed prior to transport to the cytoplasm for translation into protein at the ribosome. Such processing includes the addition of a 5′ methylated cap and the addition of a poly(A) tail to the 3′ end of the mRNA.

Maturation of 90-95% of mammalian mRNAs then occurs with splicing of the mRNA. Introns (or intervening sequences) are regions of a primary transcript (or the DNA encoding it) that are not included in the coding sequence of the mature mRNA. Exons (expressed sequences) are regions of a primary transcript (or the DNA encoding it) that remain in the mature mRNA when it reaches the cytoplasm. During the splicing process, exons in the pre-mRNA molecule are spliced together to form the mature mRNA sequence. Splice junctions, also referred to as splice sites, are utilized by cellular apparatus to determine which sequences are removed and where the ends to be joined start and stop.

Sequences on the 5′ side of the junction are called the 5′ splice site, or splice donor site, whereas sequences on the 3′ side the junction are referred to as the 3′ splice site, or the splice acceptor site. In splicing, the 3′ end of an upstream exon is joined to the 5′ end of the downstream ex on. Thus, the un-spliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Cryptic splice sites are those which are less often used but may be used when the usual splice site is blocked or unavailable. The use of different combinations of exons by the cell can result in multiple mRNA transcripts from a single gene.

In one application of antisense technology, an antisense oligonucleotide (AON) binds to a mRNA molecule transcribed from a gene of interest and inactivates (“turns off”) the mRNA by increasing its degradation and/or by preventing translation or translocation of the mRNA by steric hindrance. The end result is that expression of the corresponding gene (i.e., final production of the protein encoded by the corresponding gene) is reduced or eliminated. Alternatively, antisense technology can be used to affect splicing of a gene transcript. In this application, the antisense oligonucleotide binds to a pre-spliced RNA molecule (pre-messenger RNA or pre-mRNA) and re-directs the cellular splicing apparatus, thereby resulting in modification of the exon content of the spliced mRNA molecule. Thus, the overall sequence of a protein encoded by the modified mRNA differs from a protein translated from mRNA, the splicing of which was not altered (i.e., the full length, wild-type protein). The protein that is translated from the altered mRNA may be truncated and/or it may be missing critical sequences required for proper function. Typically, the compounds used to affect splicing are, or contain, oligonucleotides having a base sequence complementary to the mRNA being targeted. Such oligonucleotides are referred to herein as “antisense oligonucleotides” (AONs).

This disclosure provides antisense technology to modulate splicing of pre-mRNAs encoding a Kit protein, thereby causing a decrease in the amount or “level” of Kit protein expressed by a cell. Accordingly, a method of this disclosure can generally be accomplished by contacting a cell expressing a Kit transcript, with an antisense oligomer targeted to a region of the Kit pre-mRNA. Such contact results in uptake of the antisense oligomer by the cell, hybridization of the oligomer to the Kit mRNA, and subsequent modulation of splicing of the Kit pre-mRNA. In some embodiments of the presently disclosed methods, such modulation of splicing of the Kit mRNA decreases expression of Kit. The presently disclosed subject matter is not limited to the particular embodiments described herein, as such may vary. Additionally, the terminology used herein is for the purpose of describing particular embodiments only, and is not intended to be limiting on the presently disclosed subject matter.

As used herein, the term “Kit” refers to genetic loci that encode a mast/stem cell growth factor receptor (SCFR), also known as proto-oncogene c-Kit, tyrosine-protein kinase Kit, and CD117, in an animal (e.g., a mammal). The Kit protein is a receptor tyrosine kinase. Kit genes and gene products have been identified in many species, and in any given species there are generally several different gene products (e.g., RNAs) that are encoded by the Kit locus. An exemplary, non-limiting summary of Kit gene products present in the GENBANK® biosequence database is provided in Table 1.

It is understood that Table 1 includes the Accession Nos. for only certain exemplary Kit gene products in the GENBANK® biosequence database, and that other species also encode and express Kit gene products, all of which are encompassed within the scope of the presently disclosed subject matter. It is further understood that the listed species and other species can have additional Kit gene products not explicitly listed, and that all of these additional Kit gene products are also within the scope of the presently disclosed subject matter. Thus, it is expressly stated that all Kit orthologs are included within the compositions and methods of the presently disclosed subject matter.

TABLE 1
Exemplary Kit Gene Products

| | | |
|---|---|---|
| Gene Product | Nucleotide | Protein |
| Name | Accession No. | Accession No. |

Homo sapiens

| | | |
|---|---|---|
| Isoform 1 precursor | NM_000222.2 | NP_000213.1 |
| Isoform 2 precursor | NM_001093772.1 | NP_001087241.1 |
| Isoform X1 | XM_005265740.1 | XP_005265797.1 |
| Isoform X2 | XM_005265741.1 | XP_005265798.1 |
| Isoform X3 | XM_017008178.1 | XP_016863667.1 |
| Isoform X4 | XM_005265742.3 | XP_005265799.1 |
| Isoform X5 | XM_017008179.1 | XP_016863668.1 |
| Isoform X6 | XM_017008180.1 | XP_016863669.1 |

Mus musculus

| | | |
|---|---|---|
| Isoform 1 precursor | NM_001122733.1 | NP_001116205.1 |
| Isoform 2 precursor | NM_021099.3 | NP_066922.2 |
| Isoform X1 | XM_017320687.1 | XP_017176176.1 |

Felis catus

| | | |
|---|---|---|
| Kit precursor | NM_001009837.3 | NP_001009837.3 |

Canis lupus familiaris

| | | |
|---|---|---|
| Kit precursor | NM_001003181.1 | NP_001003181.1 |
| Isoform X1 | XM_005627968.3 | XP_005628025.1 |
| Isoform X2 | XM_005627969.3 | XP_005628026.1 |
| Isoform X3 | XM_005627970.3 | XP_005628027.1 |

Equus caballus

| | | |
|---|---|---|
| Kit precursor | NM_001163866.2 | NP_001157338.2 |
| Isoform X1 | XM_014738507.2 | XP_014593993.2 |
| Isoform X2 | XM_005608574.3 | XP_005608631.2 |
| Isoform X3 | XM_005608573.3 | XP_005608630.2 |

Other Organisms

| | | |
|---|---|---|
| Pan paniscus isoform X1 | XM_008954177.1 | XP_008952425.1 |
| Gorilla gorilla gorilla isoform X1 | XM_019025974.1 | XP_018881519.1 |
| Macaca mulatta Kit precursor | NM_001266095.1 | NP_001253024.1 |
| Pongo abelii isoform X1 | XM_024246425.1 | XP_024102193.1 |
| Macaca fascicularis isoform X1 | XM_005555272.2 | XP_005555329.1 |
| Sus scrofa Kit precursor | NM_001044525.1 | NP_001037990.1 |
| Rattus norvegicus Kit precursor | NM_022264.1 | NP_071600.1 |
| Bos Taurus Kit precursor | NM_001166484.1 | NP_001159956.1 |
| Gallus gallus Kit precursor | NM_204361.1 | NP_989692.1 |
| Ovis aries Kit precursor | NM_001308594.1 | NP_001295523.1 |

Similarly, a Kit coding sequence refers to a nucleic acid sequence encoding at least a portion of a Kit protein. Such a portion can be a fragment of the protein (e.g., a 10, 20, 30, 40, 50, 60, 70, 80, 90, or 100 contiguous amino acid segment from any part of the whole protein), an exon, or a domain (e.g., a transmembrane domain), or it can refer to the entire protein, including any splicing variants. c-Kit genes or coding sequences of this disclosure can be from any mammal having such gene or coding sequence. By way of example and not limitation, the c-Kit gene, coding sequence, and/or gene product may be from a human, a mouse, a canine, a feline, an equine, or another mammal. As used herein, a c-Kit transcript is an RNA molecule transcribed from a c-Kit genetic locus. In some embodiments, c-Kit transcripts targeted by oligomers of this disclosure are primary transcripts or pre-mRNA molecules. As used herein, primary mRNA or pre-mRNA is an mRNA transcript that has not yet undergone splicing. Accordingly, a mature mRNA molecule is an mRNA molecule that has undergone splicing.

As used herein, the term antisense oligomer refers to a polymeric molecule comprising nucleobases, which is capable of hybridizing to a sequence in a nucleic acid molecule, such as an mRNA molecule. The term nucleobase, as used herein, refers to the heterocyclic base portion of a nucleoside. In general, a nucleobase is any group that contains one or more atoms, or groups of atoms, capable of hydrogen bonding to a base of another nucleoside. In addition to “unmodified” or “natural” nucleobases such as the purine nucleobases adenine (A) and guanine (G), and the pyrimidine nucleobases thymine (T), cytosine (C) and uracil (U), modified nucleobases or nucleobase mimetics known to those skilled in the art are also amenable to this disclosure. The term “modified nucleobase” refers to a nucleobase that is similar in structure to the parent nucleobase, such as for example, a 7-deaza purine, a 5-methyl cytosine, a G-clamp, or a tricyclic phenoxazine nucleobase mimetic. Methods for preparation of these modified nucleobases are known to those skilled in the art. As is known in the art, a nucleoside is a base-sugar combination. The base portion of the nucleoside is normally a heterocyclic base (e.g., a nucleobase or simply a “base”). The two most common classes of such heterocyclic bases are purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage.

It is understood in the art that RNA molecules often have a short half-life, making their use as therapeutic agents problematic. Thus, chemical modifications in oligonucleotides are often included to alter their activity. Chemical modifications can alter oligomer activity by, for example, increasing affinity of an antisense oligomer for its target RNA, increasing nuclease resistance (e.g., resistance to ribonucleases such as RNaseH), and/or altering the pharmacokinetics (e.g. half-life) of the oligomer. For example, it is possible to replace sugars, nucleobases and/or internucleoside linkages with a group that maintains the ability of the oligomer to hybridize to its target sequence, but which imparts a desirable characteristic to the oligomer (e.g., resistance to degradation, increased half-life, etc.). Such groups can be referred to as analogs (e.g., sugar analog, nucleobase analog, etc.). Generally, an analog is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleotide linkage combination include, but are not limited to, peptide nucleic acids (PNA) and morpholino groups linked by uncharged, achiral linkages. In some instances, an analog is used in place of the nucleobase. Representative nucleobase mimetics are well known in the art and include, but are not limited to, tricyclic phenoxazine analogs and universal bases (see e.g., Berger et al., 2000, incorporated herein by reference). Examples of such sugar, nucleotide, and nucleobase mimetics are disclosed in U.S. Pat. Nos. 8,765,703 and 8,946,183, which are incorporated herein by reference). Methods of synthesis of sugar, nucleotide, and nucleobase mimetics, and the use of such mimetics to produce oligonucleotides are well known to those skilled in the art.

The term “oligomer” includes oligonucleotides, oligonucleosides, oligonucleotide analogs, oligonucleotide mimetics and chimeric combinations thereof. Such molecules are generally known to those skilled in the art. Oligomers of this disclosure include, but are not limited to, primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomers of this disclosure can be any length suitable for administering to a cell or individual in order to modulate splicing of an mRNA molecule. For example, antisense oligomers of this disclosure can comprise from about 10 to about 50 nucleobases (i.e., from about 10 to about 50 linked nucleosides). One having ordinary skill in the art will appreciate that this embodies antisense oligomers of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases. In some embodiments, antisense oligomers of this disclosure can comprise, or consist of, 10 to 30 nucleobases, or 10 to 25 nucleobases. Methods of determining the appropriate length for antisense oligomers of this disclosure would be apparent to those skilled in the art upon a review of this disclosure.

As used herein, the terms “targeted to”, “targeting”, and the like refer to a process of designing an antisense oligomer so that it specifically hybridizes with a desired nucleic acid molecule, such as a desired mRNA molecule. The terms “hybridizes”, “hybridization”, “hybridize to”, and the like are terms of art, and refer to the pairing of nucleobases in complementary strands of oligonucleotides (e.g., an antisense oligomer and a target sequence in a mRNA molecule). While not limited to a particular mechanism, the most common mechanism of pairing involves hydrogen bonding, which may be Watson Crick, Hoogsteen or reversed Hoogsteen hydrogen bonding, between complementary nucleoside or nucleotide bases (nucleobases). For example, the natural base adenine is complementary to the natural nucleobases thymidine and uracil, which pair through the formation of hydrogen bonds. Similarly, the natural base guanine is complementary to the natural bases cytosine and 5-methyl cytosine. In the context of this disclosure, the phrase “specifically hybridizes” refers to the capacity of an antisense oligomer of this disclosure to preferentially bind an mRNA (e.g., pre-mRNA) encoding a Kit protein rather than binding an mRNA encoding a protein unrelated in structure to a Kit protein. Further, an antisense oligomer that preferentially binds a target sequence is one that hybridizes with an mRNA encoding a Kit protein (a Kit pre-mRNA), but which does not exhibit significant hybridization with mRNA molecules encoding proteins unrelated in structure to a Kit protein. In the context used herein, significant hybridization is, for example, binding of an oligomer of this disclosure to an mRNA encoding a protein unrelated in structure to a Kit protein, with an affinity or avidity sufficiently high enough to interfere with the ability of the antisense oligomer to achieve the desired effect. Examples of such desired effects include, but are not limited to, modulation of splicing of a Kit pre-mRNA, reduction in the level of expression of Kit protein, and a reduction or inhibition in Kit-related symptoms in an individual. Thus, upon a review of the instant disclosure, it will be understood by those skilled in the art that an antisense oligomer is considered specific for a target sequence (is specifically hybridizable, specifically hybridizes, etc.) when there is a sufficient degree of complementarity between the linear sequence of nucleobases in the antisense oligomer and a linear sequence of nucleobases in the target sequence, to avoid significant binding of the antisense oligomer to non-target nucleic acid sequences under conditions in which specific binding is desired (i.e., under physiological conditions in the case of in vivo assays or therapeutic treatment, and under conditions in which assays are performed in the case of in vitro assays).

As used herein, the terms “complement”, “complementary”, “complementarity”, and the like, refer to the capacity for precise pairing between nucleobases in an oligomer and nucleobases in a target sequence. Thus, if a nucleobase (e.g., adenine) at a certain position of an oligomer is capable of hydrogen bonding with a nucleobase (e.g., uracil) at a certain position in a target sequence in a target nucleic acid, then the position of hydrogen bonding between the oligomer and the target nucleic acid is considered to be a complementary position. Usually, the terms complement, complementary, complementarity, and the like, are viewed in the context of a comparison between a defined number of contiguous nucleotides in a first nucleic acid molecule (e.g., an oligomer) and a similar number of contiguous nucleotides in a second nucleic acid molecule (e.g., a mRNA molecule), rather than in a single base to base manner. For example, if an antisense oligomer is 25 nucleotides in length, its complementarity with a target sequence is usually determined by comparing the sequence of the entire oligomer, or a defined portion thereof, with a number of contiguous nucleotides in a mRNA molecule. An oligomer and a target sequence are complementary to each other when a sufficient number of corresponding positions in each molecule are occupied by nucleobases which can hydrogen bond with each other. Positions are corresponding when the bases occupying the positions are spatially arranged such that, if complementary, the bases form hydrogen bonds. As an example, when comparing the sequence of an oligomer to a similarly sized sequence in a target sequence, the first nucleotide in the oligomer is compared with a chosen nucleotide at the start of the target sequence. The second nucleotide in the oligomer (3′ to the first nucleotide) is then compared with the nucleotide 5 directly 3′ to the chosen start nucleotide. This process is then continued with each nucleotide along the length of the oligomer. Thus, the terms “specifically hybridizable” and “complementary” are terms which are used to indicate a sufficient degree of precise pairing or complementarity over a sufficient number of contiguous nucleobases such that stable and specific binding occurs between the antisense compound and a target nucleic acid.

Hybridization conditions under which a first nucleic acid molecule will specifically hybridize with a second nucleic acid molecule are commonly referred to in the art as stringent hybridization conditions. It is understood by those skilled in the art that stringent hybridization conditions are sequence-dependent and can be different in different circumstances. Thus, stringent conditions under which an oligomer of this disclosure specifically hybridizes to a target sequence are determined by the complementarity of the oligomer sequence and the target sequence and the nature of the assays in which they are being investigated. Persons skilled in the relevant art are capable of designing complementary sequences that specifically hybridize to a particular target sequence for a given assay or a given use.

The process of designing an antisense oligomer that is targeted to a nucleic acid molecule usually begins with identification of a target nucleic acid, the expression of which is to be modulated, and determining the sequence of the target nucleic acid molecule. As used herein, the terms “target nucleic acid”, “nucleic acid encoding a Kit protein”, and the like, encompass, for example, DNA encoding a Kit protein, RNA (including pre-mRNA and mRNA) transcribed from such DNA, and cDNA derived from such RNA For example, the target nucleic acid can be a cellular gene (or pre-mRNA or mRNA transcribed therefrom), the expression of which is associated with a particular disorder or disease state. Thus, in some embodiments, a useful target nucleic acid encodes a Kit protein. In some embodiments, the target nucleic acid is a c-Kit transcript. In some embodiments, the target nucleic acid is a c-Kit pre-mRNA. Once a target nucleic acid has been identified, the targeting process includes determining at least one target region in which the antisense interaction will occur, thereby modulating splicing of the target nucleic acid. As used herein, a target region is defined as a portion of the target nucleic acid having at least one identifiable structure, function, or characteristic. Exemplary target regions are those comprising sequences involved in splicing of pre-mRNA molecules. Examples of such identifiable structures, functions, or characteristics include, but are not limited to, at least a portion of an intron or exon, an intron/exon junction, a splice donor site, a splice acceptor site, a splice branch point or a splice enhancer site. Thus, in some embodiments, the target region comprises at least part of an intron or exon, a splice donor site, a splice acceptor site, a splice branch point, and/or a splice enhancer site. In some embodiments, the target region comprises at an intron or exon, a splice donor site, a splice acceptor site, a splice branch point, and/or a splice enhancer site.

Following identification of a target region, a target sequence within the target region can then be identified. As used herein, a target sequence is a nucleic acid sequence in a target region, to which an antisense oligomer of this disclosure specifically hybridizes. Exemplary target sequences are those involved in splicing of pre-mRNA. Once a target sequence has been identified, the antisense oligomer is designed to include a nucleobase sequence sufficiently complementary to the target sequence so that the antisense oligomer specifically hybridizes to the target nucleic acid. More specifically, the nucleotide sequence of the antisense oligomer is designed so that it contains a region of contiguous nucleotides sufficiently complementary to the target sequence so that the antisense oligomer specifically hybridizes to the target nucleic acid. Such a region of contiguous, complementary nucleotides in the oligomer can be referred to as an “antisense sequence” or a “targeting sequence.”

It is well known in the art that the greater the degree of complementarity between two nucleic acid sequences, the stronger and more specific is the hybridization interaction. It is also well understood that the strongest and most specific hybridization occurs between two nucleic acid molecules that are fully complementary. As used herein, the term fully complementary refers to a situation when each nucleobase in a nucleic acid sequence is capable of hydrogen binding with the nucleobase in the corresponding position in a second nucleic acid molecule. In some embodiments, the targeting sequence is fully complementary to the target sequence. In some embodiments, the targeting sequence comprises an at least 6 contiguous nucleobase region that is fully complementary to an at least 6 contiguous nucleobase region in the target sequence. In some embodiments, the targeting sequence comprises an at least 8 contiguous nucleobase sequence that is fully complementary to an at least 8 contiguous nucleobase sequence in the target sequence. In some embodiments, the targeting sequence comprises an at least 10 contiguous nucleobase sequence that is fully complementary to an at least 10 contiguous nucleobase sequence in the target sequence. In some embodiments, the targeting sequence comprises an at least 12 contiguous nucleobase sequence that is fully complementary to an at least 12 contiguous nucleobase sequence in the target sequence. In some embodiments, the targeting sequence comprises an at least 14 contiguous nucleobase sequence that is fully complementary to an at least 14 contiguous nucleobase sequence in the target sequence. In some embodiments, the targeting sequence comprises an at least 16 contiguous nucleobase sequence that is fully complementary to an at least 16 contiguous nucleobase sequence in the target sequence. In some embodiments, the targeting sequence comprises an at least 18 contiguous nucleobase sequence that is fully complementary to an at least 18 contiguous nucleobase sequence in the target sequence. In some embodiments, the targeting sequence comprises an at least 20 contiguous nucleobase sequence that is fully complementary to an at least 20 contiguous nucleobase sequence in the target sequence.

It will be understood by those skilled in the art that the targeting sequence may make up the entirety of an antisense oligomer of this disclosure, or it may make up just a portion of an antisense oligomer of this disclosure. For example, in an oligomer consisting 20 of 30 nucleotides, all 30 nucleotides can be complementary to a 30 contiguous nucleotide target sequence. Alternatively, for example, only 20 contiguous nucleotides in the oligomer may be complementary to a 20-contiguous nucleotide target sequence, with the remaining 10 nucleotides in the oligomer being mismatched to nucleotides outside of the target sequence. In some embodiments, oligomers of this disclosure have a targeting sequence of at least 10 nucleobases, at least 11 nucleobases, at least 12 nucleobases, at least 13 nucleobases, at least 14 nucleobases, at least 15 nucleobases, at least 16 nucleobases, at least 17 nucleobases, at least 18 nucleobases, at least 19 nucleobases, at least 20 nucleobases, at least 21 nucleobases, at least 22 nucleobases, at least 23 nucleobases, at least 24 nucleobases, at least 25 nucleobases, at least 26 nucleobases, at least 27 nucleobases, at least 28 nucleobases, at least 29 nucleobases, or at least 30 nucleobases in length.

It will be understood by those skilled in the art that the inclusion of mismatches between a targeting sequence and a target sequence is possible without eliminating the activity of the oligomer (e.g., modulation of splicing). Moreover, such mismatches can occur anywhere within the antisense interaction between the targeting sequence and the target sequence, so long as the antisense oligomer is capable of specifically hybridizing to the targeted nucleic acid molecule. Thus, antisense oligomers of this disclosure may comprise up to about 20% nucleotides that are mismatched, thereby disrupting base pairing of the antisense oligomer to a target sequence, as long as the antisense oligomer specifically hybridizes to the target sequence. In some embodiments, antisense oligomers comprise no more than 20%, no more than about 15%, no more than about 10%, no more than about 5% or not more than about 3% of mismatches, or less. In some embodiments, there are no mismatches between nucleotides in the antisense oligomer involved in pairing and a complementary target sequence. In some embodiments, mismatches do not occur at contiguous positions. For example, in an antisense oligomer containing 3 mismatch positions, in some embodiments the mismatched positions can be separated by runs (e.g., 3, 4, 5, etc.) of contiguous nucleotides that are complementary with 15 nucleotides in the target sequence.

The use of percent identity is a common way of defining the number of mismatches between two nucleic acid sequences. For example, two sequences having the same nucleobase pairing capacity would be considered 100% identical. Moreover, it should be understood that both uracil and thymidine will bind with adenine. Consequently, two molecules that are otherwise identical in sequence would be considered identical, even if one had uracil at position x and the other had a thymidine at corresponding position x. Percent identity may be calculated over the entire length of the oligomeric compound, or over just a portion of an oligomer. For example, the percent identity of a targeting sequence to a target sequence can be calculated to determine the capacity of an oligomer comprising the targeting sequence to bind to a nucleic acid molecule comprising the target sequence. In some embodiments, the targeting sequence is at least 80% identical, at least 85% identical, at least 90% identical, at least 95% identical, at least 97% identical, at least 98% identical or at least 99% identical over its entire length to a target sequence in a target nucleic acid molecule. In some embodiments, the targeting sequence is identical over its entire length to a target sequence in a target nucleic acid molecule. It is understood by those skilled in the art that an antisense oligomer need not be identical to the oligomer sequences disclosed herein to function similarly to the antisense oligomers described herein. Shortened versions of antisense oligomers taught herein, or non-identical versions of the antisense oligomers taught herein, fall within the scope of this disclosure. Non-identical versions are those wherein each base does not have 100% identity with the antisense oligomers disclosed herein. Alternatively, a non-identical version can include at least one base replaced with a different base with different pairing activity (e.g., G can be replaced by C, A, or T). Percent identity is calculated according to the number of bases that have identical base pairing corresponding to the oligomer to which it is being compared. The non-identical bases may be adjacent to each other, dispersed throughout the oligomer, or both. For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art. Thus, antisense oligomers of this disclosure comprise oligonucleotide sequences at least 80% identical, at least 85% identical, at least 90% identical, at least 92% identical, at least 94% identical at least 96% identical or at least 98% identical to sequences disclosed herein, as long as the antisense oligomers are able to modulate splicing of a desired mRNA molecule.

Antisense oligomers of this disclosure are capable of modulating splicing of mRNA molecules. As used herein, “modulation” of splicing refers to the ability of an antisense oligomer to affect the processing of a pre-mRNA transcript such that the resulting spliced mRNA molecule contains a desired combination of exons as a result of exon skipping (or exon inclusion), a deletion in one or more exons, or additional sequence not normally found in the spliced mRNA (e.g., intronic sequences). For example, modulation of splicing can refer to affecting the splicing of a Kit pre-mRNA such that the spliced mRNA (mature mRNA) is missing at least a portion, or the entirety, of one exon. In some embodiments, the spliced mRNA lacks at least a portion of exon 4. It can be the case that a truncated isoform of the Kit protein is present in cells, and that such truncation is due to a truncation in exon 4 of the mRNA encoding the Kit protein. Thus, for the purposes of describing this disclosure, splicing of a Kit pre-mRNA, due to the influence of an antisense oligomer, to produce a truncated mRNA encoding a truncated Kit protein, can be referred to as alternative splicing. Further, a Kit mRNA transcript lacking at least a portion, or the entirety, of exon 4, due to the influence of an antisense oligomer, is a product of alternative splicing. Thus, in the context of this disclosure, modulation of splicing can refer to inducing alternative splicing of a Kit pre-mRNA molecule, thereby reducing the level of mRNA molecules containing the entirely of exon 4, and increasing the level of mRNA molecules lacking at least a portion of exon 4.

Although the present disclosure exemplifies the compositions and methods with respect to targeting exon 4 of the human or murine Kit gene, it is understood that both other exons of the human or murine Kit gene could also be targeted in humans and mice. Additionally, exon 4 or any other exon could also be targeted in cats, dogs, horses, or other animals, and thus the particular examples of targeting strategies disclosed herein is understood to be exemplary only. A summary of the genomic organization of the Kit locus in particular exemplary animals is provided in Table 2.

TABLE 2
Genomic Organization of Kit Loci in Exemplary Animals

| | Genomic | |
|---|---|---|
| | Locus | |
| | SEQ ID | Nucleotide Positions of |
| Species | NO: | Exons in Genomic Locus |
| Homo | 18 | 1-154; 37585-37854; 40357-40638; |
| sapiens | | 41703-41839; 45797-45965; |
| | | 49171-49360; 51497-51612; 65657- |
| | | 65771; 67930-68123; 69291-69397; |
| | | 69489-69615; 69896-70000; |
| | | 70084-70194; 71408-71558; 73401- |
| | | 73492; 73944-74071; 75143-75265; |
| | | 78571-78682; 78794-78893; |
| | | 79248-79353; 80502-82788 |
| Mus | 19 | 1-132; 32040-32312; 34284-34565; |
| musculus | | 35821-35957; 40333-40507; |
| | | 45861-46050; 48003-48118; 62308- |
| | | 62422; 63979-64172; 65507-65613; |
| | | 65713-65839; 66124-66228; |
| | | 66341-66451; 70850-71000; 72762- |
| | | 72850; 73401-73528; 74561-74683; |
| | | 77564-77675; 77771-77870; |
| | | 78234-78339; 79428-81736 |
| Felis | 20 | Reverse complement of 1-1102; |
| catus | | 2207-2312; 2677-2776; 2888-2999; |
| | | 7017-7139; 8196-8323; 8814- |
| | | 8905; 10742-10892; 12058-12168; |
| | | 12258-12362; 12645-12771; |
| | | 12869-12975; 14198-14391; 15933- |
| | | 16047; 31125-31240; 33427-33616; |
| | | 38728-38899; 43910-44047; |
| | | 45147-45428; 48253-48522; 86237- |
| | | 86372 |
| Canis lupus | 21 | 1-101; 35944-36213; 38998-39279; |
| familiaris | | 40364-40500; 45080-45251; |
| | | 49157-49346; 52424-52539; 66555- |
| | | 66669; 68419-68612; 69809-69915; |
| | | 70009-70135; 70421-70525; |
| | | 70612-70722; 71913-72063; 73946- |
| | | 74037; 74515-74642; 75747-75869; |
| | | 79360-79471; 79585-79684; |
| | | 80038-80143; 81200-81517 |
| Equus | 22 | Reverse complement of 1-129; |
| caballus | | 1273-1378; 1734-1833; 1950-2061; |
| | | 5375-5497; 6550-6677; 7176- |
| | | 7267; 9440-9590; 10826-10936; |
| | | 11023-11127; 11397-11523; |
| | | 11617-11723; 13186-13367; 15056- |
| | | 15170; 28125-28240; 30357-30546; |
| | | 34989-35160; 39789-39925; |
| | | 41022-41303; 43889-44158; 79800- |
| | | 79957 |

With respect to Table 2, since the Table lists the positions of the exons in SEQ ID NOs: 18-22, it is understood that the nucleotides that fall between the listed positions are intron sequences. For example, the human c-KIT exon sequences are listed as corresponding to nucleotides 1-154, 37585-37854, 40357-40638, 41703-41839, 45797-45965, 49171-49360, 51497-51612, 65657-65771, 67930-68123, 69291-69397, 69489-69615, 69896-70000, 70084-70194, 71408-71558, 73401-73492, 73944-74071, 75143-75265, 78571-78682, 78794-78893, 79248-79353, and 80502-82788 of SEQ ID NO: 18. Since SEQ ID NO: 18 is a genomic sequence, it follows that the human c-KIT intron sequences correspond to nucleotides 155-37584, 37855-40356, 40639-41702, 41840-45796, 45966-49170, 49361-51496, 51613-65656, 65772-67929, 68124-69290, 69398-69488, 69616-69895, 70001-70083, 70195-71407, 71557-73400, 73493-73943, 74072-75142, 75266-78570, 78683-78793, 78894-79247, and 79354-80501 of SEQ ID NO: 18.

In designing an ESO, in some embodiments the ESO spans an exon/intron or intron/exon boundary, meaning that the ESO comprises contiguous nucleotides that are both 5′ and 3′ of a splice donor or a splice acceptor. The number of nucleotides of the ESO that are part of an exon and the number of nucleotides of the ESO that are part of the adjacent intron can vary, but in some embodiments, there are at least 5 exon nucleotides and at least 5 intron nucleotides. By way of example and not limitation, if the ESO is designed to have 25 nucleotides as in the cases of SEQ ID NOs: 1 and 2, in some embodiments the ESO includes 5, 6, 7, 8, 9, 10, 11 12, 13, 14, 15, 16, 17, 18, 19, or 20 contiguous exon nucleotides and also includes the directly adjacent 10, 19, 18, 17, 16, 15, 14, 13, 12, 11, 10, 9, 8, 7, 6, or 5 contiguous intron nucleotides, respectively. Stated another way, for any of SEQ ID NOs: 18-22, an ESO can comprise a sequence of in some embodiments 15, 16, 17, 18, 19, 20, 21, 22, 23, 24, 25, or more contiguous nucleotides of any of SEQ ID NOs: 18-22, which 15-25 or more contiguous nucleotides include at least one splice donor or splice acceptor sequence. Extensions of the sequences in the 5′ and/or 3′ directions can also occur such that ESOs can be designed to target pre-mRNA sequences of 26, 27, 28, 29, 30, 31, 32, 33, 34, 35, 36, 37, 38, 39, 40, 41, 42, 43, 44, 45, 46, 47, 48, 49, 50, or more nucleotides in length.

Exemplary target sequences that can be employed to design ESOs for use in targeting human c-KIT gene products are presented in Table 3. It is noted that target sequences that can be employed to design ESOs for use in targeting c-Kit gene products in non-human animals can be based on the approaches taken for the exemplary ESOs in Table 3.

TABLE 3
Exemplary Target Sequences for
Targeting Human c-KIT Gene Products

| Exon | | Donor Target Sequence* |
|---|---|---|
| 2 | Acceptor** | aacacgattctgtttttcttggcagGCTCTTCTCAACCATCTGTGAGTCC |
| | | (SEQ ID NO: 23; nucleotides 37560-37609 of SEQ ID NO: 18) |
| | Donor | AATTCCATTTATGTGTTTGTTAGAGgtaaatgcttggctttctgcagtgc |
| | | (SEQ ID NO: 24; nucleotides 37830-37879 of SEQ ID NO: 18) |
| 3 | Acceptor | actgattttggatatgcttctatagATCCTGCCAAGCTTTTCCTTGTTGA |
| | | (SEQ ID NO: 25; nucleotides 40332-40381 of SEQ ID NO: 18) |
| | Donor | AAATTCATCCTGAAAGTGAGGCCAGgtactggctctttcttatctgcctc |
| | | (SEQ ID NO: 26; nucleotides 40614-40663 of SEQ ID NO: 18) |
| 4 | Acceptor | ggggccacatttcttttcattctagCCTTCAAAGCTGTGCCTGTTGTGTC |
| | | (SEQ ID NO: 27; nucleotides 41678-41727 of SEQ ID NO: 18) |
| | Donor | AACGTGGAAAAGAGAAAACAGTCAGgtgagtgaatcgcttcattcttctc |
| | | (SEQ ID NO: 28; nucleotides 41815-41864 of SEQ ID NO: 18) |
| 5 | Acceptor | ttttctccttttctgaaaccagcagACTAAACTACAGGAGAAATATAATA |
| | | (SEQ ID NO: 29; nucleotides 45772-45821 of SEQ ID NO: 18) |
| | Donor | GTCACAACAACCTTGGAAGTAGTAGgtaaatacctctatgggaatgttta |
| | | (SEQ ID NO: 30; nucleotides 45941-45990 of SEQ ID NO: 18) |
| 6 | Acceptor | ttctgtcttatttcattctaattagATAAAGGATTCATTAATATCTTCCC |
| | | (SEQ ID NO: 31; nucleotides 49146-49195 of SEQ ID NO: 18) |
| | Donor | AGTCTGAGAATGAAAGTAATATCAGgtaagaaatggaccttgccctgggg |
| | | (SEQ ID NO: 32; nucleotides 49336-49385 of SEQ ID NO: 18) |
| 7 | Acceptor | ttgactagttgtcttttctttgtagATACGTAAGTGAACTTCATCTAACG |
| | | (SEQ ID NO: 33; nucleotides 51472-51521 of SEQ ID NO: 18) |
| | Donor | ATAGCATTTAATGTTTATGTGAATAgtaagtaacatgaagggctcctttt |
| | | (SEQ ID NO: 34; nucleotides 51588-51637 of SEQ ID NO: 18) |
| 8 | Acceptor | tatggccatttctgttttcctgtagCAAAACCAGAAATCCTGACTTACGA |
| | | (SEQ ID NO: 35; nucleotides 65632-65681 of SEQ ID NO: 18) |
| | Donor | ATTTTTGTCCAGGAACTGAGCAGAGgtgagatgattatttttggcactgc |
| | | (SEQ ID NO: 36; nucleotides 65747-65796 of SEQ ID NO: 18) |
| 9 | Acceptor | ggcttttgttttcttccctttagATGCTCTGCTTCTGTACTGCCAGTG |
| | | (SEQ ID NO: 37; nucleotides 67907-67954 of SEQ ID NO: 18) |
| | Donor | TTTGCATTTAAAGGTAACAACAAAGgtatatttctttttaatccaattta |
| | | (SEQ ID NO: 38; nucleotides 68099-38148 of SEQ ID NO: 18) |
| 10 | Acceptor | attccacatttctcttccattgtagAGCAAATCCATCCCCACACCCTGTT |
| | | (SEQ ID NO: 39; nucleotides 69266-29315 of SEQ ID NO: 18) |
| | Donor | GATTCTGACCTACAAATATTTACAGgtaaccatttatttgttctctctcc |
| | | (SEQ ID NO: 40; nucleotides 69373-69422 of SEQ ID NO: 18) |
| 11 | Acceptor | tctatttttccctttctccccacagAAACCCATGTATGAAGTACAGTGGA |
| | | (SEQ ID NO: 41; nucleotides 69464-69513 of SEQ ID NO: 18) |
| | Donor | TTTCCCAGAAACAGGCTGAGTTTTGgtcagtatgaaacaggggctttcca |
| | | (SEQ ID NO: 42; nucleotides 69591-69640 of SEQ ID NO: 18) |
| 12 | Acceptor | taccttgttgtcttccttcctacagGGAAAACCCTGGGTGCTGGAGCTTT |
| | | (SEQ ID NO: 43; nucleotides 69871-69920 of SEQ ID NO: 18) |
| | Donor | ACTGTCGCTGTAAAGATGCTCAAGCgtaagttcctgtatggtactgcatg |
| | | (SEQ ID NO: 44; nucleotides 69976-70025 of SEQ ID NO: 18) |
| 13 | Acceptor | ctaaaatgcatgtttccaattttagCGAGTGCCCATTTGACAGAACGGGA |
| | | (SEQ ID NO: 45; nucleotides 70059-70108 of SEQ ID NO: 18) |
| | Donor | CTACTTGGAGCCTGCACCATTGGAGgtaaagccgtgtccaagctgccttt |
| | | (SEQ ID NO: 46; nucleotides 70170-70219 of SEQ ID NO: 18) |
| 14 | Acceptor | ctaaccttttcttatgtgcttttagGGCCCACCCTGGTCATTACAGAATA |
| | | (SEQ ID NO: 47; nucleotides 71383-71432 of SEQ ID NO: 18) |
| | Donor | TTCTGCATTCAAAGGAGTCTTCCTGgtaagactgatttacataaatagtt |
| | | (SEQ ID NO: 48; nucleotides 71534-71583 of SEQ ID NO: 18) |
| 15 | Acceptor | tcatgacttgtttcatctctcccagCAGCGATAGTACTAATGAGTACATG |
| | | (SEQ ID NO: 49; nucleotides 73376-73425 of SEQ ID NO: 18) |
| | Donor | GACAAAAGGAGATCTGTGAGAATAGgtgagtacctacctatcaagcaacc |
| | | (SEQ ID NO: 50; nucleotides 73468-73517 of SEQ ID NO: 18) |
| 16 | Acceptor | aagaaaaatcctctcttcctcacagGCTCATACATAGAAAGAGATGTGAC |
| | | (SEQ ID NO: 51; nucleotides 73919-73968 of SEQ ID NO: 18) |
| | Donor | CATGGCTTTCCTCGCCTCCAAGAATgtaagtgggagtgattctctaaaga |
| | | (SEQ ID NO: 52; nucleotides 74047-74096 of SEQ ID NO: 18) |
| 17 | Acceptor | tttcttttctcctccaacctaatagTGTATTCACAGAGACTTGGCAGCCA |
| | | (SEQ ID NO: 53; nucleotides 75118-75167 of SEQ ID NO: 18) |
| | Donor | TTCTAATTATGTGGTTAAAGGAAACgtgagtacccattctctgcttgaca |
| | | (SEQ ID NO: 54; nucleotides 75241-75290 of SEQ ID NO: 18) |
| 18 | Acceptor | tgactctgttgtgcttctattacagGCTCGACTACCTGTGAAGTGGATGG |
| | | (SEQ ID NO: 55; nucleotides 78546-78595 of SEQ ID NO: 18) |
| | Donor | TTTCTTTGGGAGCTGTTCTCTTTAGgtaaaatgatccttgccaaagacaa |
| | | (SEQ ID NO: 56; nucleotides 78658-78707 of SEQ ID NO: 18) |
| 19 | Acceptor | cactgctttgcaaactgtgtctcagGAAGCAGCCCCTATCCTGGAATGCC |
| | | (SEQ ID NO: 57; nucleotides 78769-78818 of SEQ ID NO: 18) |
| | Donor | GCCCTGAACACGCACCTGCTGAAATgtaagagccaaaaaatttttccttt |
| | | (SEQ ID NO: 58; nucleotides 78869-78918 of SEQ ID NO: 18) |
| 20 | Acceptor | atagtaaatggcccttgtcttgcagGTATGACATAATGAAGACTTGCTGG |
| | | (SEQ ID NO: 59; nucleotides 79223-79272 of SEQ ID NO: 18) |
| | Donor | GCAGATTTCAGAGAGCACCAATCATgtgagtataccctggccaggcatag |
| | | (SEQ ID NO: 60; nucleotides 79329-79378 of SEQ ID NO: 18) |

*the ESOs that target these sequences would be the reverse complement of the sequences shown or a subsequence thereof.
**“Acceptor” refers to a target sequence that includes a splice acceptor, and “Donor” refers to a target sequence that includes a splice donor for the listed exon.

In the listed sequences themselves, intron sequences are provided in lowercase and exon sequences are provided in uppercase.

In some embodiments of the presently disclosed subject matter provided is an antisense oligomer comprising 10 to 50 linked nucleosides, wherein the oligomer is targeted to a region of an RNA molecule encoding a Kit protein. In some embodiments, hybridization of the oligomer to the RNA molecule modulates splicing of the RNA molecule. In some embodiments of the presently disclosed subject matter provided is an antisense oligomer comprising a nucleic acid sequence sufficiently complementary to a target sequence in a target region of a Kit mRNA molecule, such that the antisense oligomer specifically hybridizes to the target sequence, thereby modulating splicing of a Kit mRNA transcript. These antisense oligomers may comprise, consist essentially of, or consist of 10 to 50 linked nucleosides. These antisense oligomers may comprise 15 to 35 linked nucleotides. These antisense oligomers may consist essentially of or consist of 15 to 35 linked nucleosides. These antisense oligomers may comprise, consist essentially of, or consist of 10 linked nucleosides, 11 linked nucleosides, 12 linked nucleosides, 13 linked nucleosides, 14 linked nucleosides, 15 linked nucleosides, 16 linked nucleosides, 17 linked nucleosides, 18 linked nucleosides, 19 linked nucleosides, 20 linked nucleosides, 21 linked nucleosides, 22 linked nucleosides, 23 linked nucleosides, 24 linked nucleosides, 25 linked nucleosides, 26 linked nucleosides, 27 linked nucleosides, 28 linked nucleosides, 29 linked nucleosides, 30 linked nucleosides, 31 linked nucleosides, 32 linked nucleosides, 33 linked nucleosides, 34 linked nucleosides, 34 linked nucleosides, 36 linked nucleosides, 37 linked nucleosides, 38 linked nucleosides, 39 linked nucleosides, 40 linked nucleosides, 41 linked nucleosides, 42 linked nucleosides, 43 linked nucleosides, 44 linked nucleosides, 45 linked nucleosides, 46 linked nucleosides, 47 linked nucleosides, 48 linked nucleosides, 49 linked nucleosides, or 50 linked nucleosides. The mRNA molecule may encode a Kit protein from any mammal that produces a Kit protein. Examples of such mammals include, but are not limited to, a human, a mouse, a dog, a cat, and a horse. In some embodiments, the mRNA comprises a nucleotide sequence at least 80%, at least 85%, at least 90%, at least 95%, at least 97%, at least 99%, or 100% identical to SEQ ID NO: 8 or SEQ ID NO: 10. In some embodiments, the mRNA encodes a protein comprising an amino acid sequence at least 80%, at least 85%, at least 90%, at least 95%, at least 97%, at least 99% identical to SEQ ID NO: 9 or SEQ ID NO: 11. In some embodiments, the mRNA encodes a protein comprising SEQ ID NO: 9 or SEQ ID NO: 11. The RNA molecule may be a Kit transcript. In some embodiments, the RNA molecule is a Kit mRNA molecule. In some embodiments, the RNA molecule is a Kit pre-mRNA.

The target region targeted by the antisense oligomer can be any region of the RNA molecule that is functionally involved in splicing of the RNA molecule. By “functionally involved in splicing” is meant the sequences in the target region are utilized by the cellular splicing apparatus (e.g., the spliceosome or components thereof) to effect splicing of the mRNA molecule. Examples of such regions include, but are not limited to, regions comprising intron sequences, regions comprising exon sequences, regions comprising intron/exon junctions, regions comprising splice donor site sequences, regions comprising to splice acceptor site sequences, regions comprising splice enhancer site sequences, regions comprising branch point sequences, and regions comprising polypyrimidine tracts. Such sequences are known to those skilled in the art. Such sequences are also disclosed herein. Thus, in some embodiments, the target region comprises at least a portion of a sequence selected from the group consisting of an exon sequence, an intron sequence, a sequence comprising an exon/intron junction, a splice donor site sequence, a splice acceptor site sequence, a splice enhancer site sequence, a branch point sequence, and a polypyrimidine tract. In the context of this disclosure, “at least a portion” refers to at least 5 nucleosides, at least 6 nucleosides, at least 7 nucleosides, at least 8 nucleosides, at least 9 nucleosides, at least 10 nucleosides, at least 11 nucleotides, at least 12 nucleosides, at least 13 nucleotides, at least 14 nucleosides, at least 15 nucleosides, at least 16 nucleosides, at least 17 nucleosides, at least 18 nucleosides, at least 19 nucleosides, or at least 20 nucleosides in length. In some embodiments, the at least a portion comprises at least 10%, at least 25%, at least 50%, at least 75%, at least 90%, at least 90%, at least 95% or at least 97% of a known splice donor site sequence, splice acceptor site sequence, splice enhancer site sequence, branch point sequence or polypyrimidine sequence. The splice donor site sequence, splice acceptor site sequence, splice enhancer site sequence, branch point sequence or polypyrimidine sequence may be from a Kit pre-MRNA.

In some embodiments, the target region comprises at least a portion of a Kit sequence of this disclosure, which may be any one of SEQ ID NOs: 8, 10, 12, 14, and 16 or a subsequence of any one of SEQ ID NOs: 18-22. In some embodiments, the at least a portion comprises at least 10%, at least 25%, at least 50%, at least 75%, at least 80%, at least 90% at least 95% or at least 97% of a Kit sequence of this disclosure. In some embodiments, the at least a portion comprises a polynucleotide sequence at least 80%, at least 90% at least 95% or at least 97% identical to a portion of a Kit sequence of this disclosure. In some embodiments, the target region comprises a nucleotide sequence at least 80%, at least 90%, at least 95%, at least 97%, at least 99%, or 100% identical to at least a portion of a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, or SEQ ID NO: 22. In some embodiments, the target region comprises at least a portion of a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, or SEQ ID NO: 22.

In some embodiments, the antisense oligomer is targeted to a region or sequence involved in splicing of a Kit pre-mRNA. In some embodiments, the antisense oligomer is targeted to a Kit intron sequence, a Kit exon sequence, a Kit splice donor site sequence, a Kit splice acceptor site sequence, a Kit splice enhancer site sequence, a Kit branch point sequence, or a Kit polypyrimidine tract. In some embodiments, the antisense oligomer is targeted to exon 4 of a Kit pre-mRNA. In some embodiments, the antisense oligomer is targeted to a Kit exon 4 splice donor sequence, a Kit exon 4 splice acceptor sequence, or a Kit exon 4 spice enhancer sequence. In some embodiments, the antisense oligomer is targeted to a target molecule comprising a sequence at least 80%, at least 90%, at least 95%, at least 97%, at least 99%, or 100% identical to a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, and SEQ ID NO: 22, or a subsequence thereof. In some embodiments, the antisense oligomer is targeted to a target molecule comprising a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, and SEQ ID NO: 22, or a subsequence thereof. In some embodiments, the antisense oligomer is targeted to a sequence at least 80%, at least 90%, at least 95%, at least 97%, at least 99%, or 100% identical to a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, and SEQ ID NO: 22, or a subsequence thereof.

In some embodiments, the antisense oligomer comprises a targeting sequence at least 80%, at least 90%, at least 95%, at least 97%, or at least 99% identical to a sequence fully complementary to at least a portion of a splice donor site sequence, a splice acceptor site sequence, a splice enhancer site sequence, a branch point sequence or a polypyrimidine sequence from a Kit mRNA. In some embodiments, the antisense oligomer comprises a targeting sequence fully complementary to at least a portion of a splice donor site sequence, a splice acceptor site sequence, a splice enhancer site sequence, a branch point sequence, or a polypyrimidine sequence from a Kit mRNA. In some embodiments, the antisense oligomer comprises a targeting sequence at least 80%, at least 90%, at least 95%, at least 97%, at least 99%, or 100% identical to a sequence fully complementary to at least a portion of a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, and SEQ ID NO: 22, or a subsequence thereof. The portion is in some embodiments least 10 nucleotides in length. The antisense oligomer may modulate splicing of a Kit pre-mRNA molecule.

In some embodiments, the target region comprises at least a portion of a sequence selected from a Kit splice donor site sequence, a Kit splice acceptor site sequence, a Kit splice enhancer site sequence, a Kit branch point sequence and a Kit polypyrimidine sequence. In some embodiments, the at least a portion comprises at least 10%, at least 25%, at least 50%, at least 75%, at least 90% or at least 90% of a Kit splice donor site sequence, a Kit splice acceptor site sequence, a Kit splice enhancer site sequence, a Kit branch point sequence or a Kit polypyrimidine sequence. The Kit splice donor site sequence, the Kit splice acceptor site sequence, the Kit splice enhancer site sequence, the Kit branch point sequence, or the Kit polypyrimidine sequence, may be from exon 4 of a Kit pre-mRNA. The portion may be at least 10 nucleotides in length. The antisense oligomer may modulate splicing of a Kit pre-mRNA molecule.

In some embodiments, the complementary nucleic acid sequence comprised by the antisense oligomer (i.e., the “targeting sequence”) is at least 80%, at least 90%, at least 95%, at least 97%, or at least 99% identical to a sequence fully complementary to at least a portion of a splice donor site sequence, splice acceptor site sequence, splice enhancer site sequence, branch point sequence or polypyrimidine sequence from a Kit mRNA. In some embodiments, the complementary nucleic acid sequence comprised by the antisense oligomer comprises a sequence at least 80%, at least 90%, at least 95%, at least 97%, at least 99%, or 100% identical to a sequence fully complementary to a portion of a sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 19, SEQ ID NO: 20, SEQ ID NO: 21, and SEQ ID NO: 22, or a subsequence thereof. The portion may be at least 10 nucleotides in length. The antisense oligomer may modulate splicing of a Kit pre-mRNA molecule.

In some embodiments, an antisense oligomer comprises a sequence at least 80% identical, at least 85% identical, at least 90% identical, at least 95% identical, at least 97% identical, or at least 99% identical to a sequence selected from the group consisting of SEQ ID NOs: 1 and 2. In some embodiments, an antisense oligomer comprises, consists essentially of, or consists of a sequence selected from the group consisting of SEQ ID NO: 1 and 2.

In some embodiments of the presently disclosed subject matter provided is an expression vector that expresses an antisense oligomer of this disclosure. As used herein, an “expression vector” is a nucleic acid molecule comprising a polynucleotide sequence functionally linked to a promoter, such that transcription of the polynucleotide sequence by a polymerase results in production of an antisense oligomer of this disclosure. Exemplary expression vectors include polynucleotide molecules, in some embodiments DNA molecules, which are derived, for example, from a plasmid, bacteriophage, yeast or virus (e.g., adenovirus, adeno-associated virus, lentivirus, retrovirus, etc.), into which a polynucleotide can be inserted or cloned. Suitable expression vectors are known to those skilled in the art.

In some embodiments, the presently disclosed subject matter provides a pharmaceutical composition comprising an antisense oligomer or expression vector of this disclosure. Such compositions are suitable for the therapeutic delivery of antisense oligomers, or expression vectors, described herein. Hence, this disclosure provides pharmaceutical compositions that comprise a therapeutically-effective amount of one or more of the antisense oligomers or expression vectors described herein, formulated together with one or more pharmaceutically-acceptable carriers (additives) and/or diluents. While it is possible for an antisense oligomer or expression vector of this disclosure to be administered alone, in some embodiments an antisense oligomer or expression vector of the presently disclosed subject matter is administered as a pharmaceutical composition.

Pharmaceutical compositions of this disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) inhaled into the lungs, for example, by nebulizer or aerosol inhaler; or (9) nasally. Examples of suitable carriers, additives and diluents are described in U.S. Patent Publication No. 2015/0361428, which is incorporated herein by reference in its entirety.

As has been described above, antisense oligomers of this disclosure are capable of reducing expression of Kit. Such reduction is achieved by modulating splicing of an mRNA molecule encoding a Kit protein.

Thus, in some embodiments of the presently disclosed subject matter provided is a method of modulating splicing of a Kit mRNA in a cell, the method comprising contacting the cell with an antisense oligomer of this disclosure. The cell may be any cell expressing a Kit mRNA molecule. Accordingly, the cell can be a cell in culture, or a cell in the body of an individual. In a representative embodiment, the cell is a mast cell.

In some embodiments of the presently disclosed subject matter provided is a method of inducing apoptosis in mast cells, the method comprising contacting the mast cell with the antisense oligomer of this disclosure.

Thus, in some embodiments of the presently disclosed subject matter provided is a method of treating a Kit-related disease in an individual, the method comprising administering an antisense oligomer of this is disclosure. In some embodiments, the Kit-related disease is a cancer or mastocytosis. In some embodiments, the cancer is a gastrointestinal stromal tumor or leukemia.

Mastocytosis is a rare mast cell activation disorder caused by an individual having too many mast cells and mast cell precursors. Because mast cells are involved in atopic responses, individuals suffering from mastocytosis are susceptible to hives, itching and anaphylactic shock. Thus, one method of this disclosure is a method of treating an individual suffering from mastocytosis, the method comprising administering to an individual in need of such treatment an antisense oligomer of this disclosure. The individual may or may not already be exhibiting symptoms of mastocytosis, such as itching, hives and anaphylaxis. In some embodiments, an antisense oligomer is administered to an individual at risk for developing symptoms of mastocytosis.

The mRNA may comprise a nucleotide sequence at least 80%, at least 85%, at least 90%, at least 95%, at least 97%, at least 99%, or 100% identical to SEQ ID NO: 8, 10, 12, 14, or 16. In some embodiments, the mRNA encodes a protein comprising an amino acid sequence at least 80%, at least 85%, at least 90%, at least 95%, at least 97%, or at least 99% identical to SEQ ID NO: 9, 11, 13, 15, or 17. In some embodiments, the mRNA encodes a protein comprising SEQ ID NO: 9, 11, 13, 15, or 17.

In some embodiments, the antisense oligomer hybridizes to a target region that is involved in splicing of Kit pre-mRNA. In some embodiments, the antisense oligomer hybridizes to a target region in the mRNA comprising at least a portion of a sequence selected from the group consisting of a Kit splice donor site sequence, a Kit splice acceptor site sequence, a Kit splice enhancer site sequence, a Kit branch point sequence and a Kit polypyrimidine sequence. The Kit splice donor site sequence, the Kit splice acceptor site sequence, the Kit splice enhancer site sequence, the Kit branch point sequence, or the Kit polypyrimidine sequence may be from exon 4 of a Kit pre-mRNA.

In some embodiments of the presently disclosed subject matter provided is a method of reducing expression of Kit protein in a cell, the method comprising contacting the cell with an antisense oligomer of this disclosure. In embodiments of this disclosure, the cell can be any cell expressing a Kit protein. Accordingly, the cell can be a cell in culture (e.g., tissue culture) or a cell in the body of an individual. In some embodiments, the amount of Kit expressed by the cell is decreased by at least 50%, at least 60%, at least 70%, at least 80%, at least 90%, at least 95%, at least 97%, or at least 99%. In a representative embodiment, the cell is a mast cell.

An antisense oligomer of this disclosure may be administered to any individual expressing a Kit protein. As used herein, the terms individual, subject, patient, and the like, are meant to encompass any mammal that expresses a Kit protein, such as but not limited to a human, a mouse, a cat, a dog, or a horse. The terms individual, subject, and patient by themselves do not denote a particular age, sex, race, and the like. Thus, individuals of any age, whether male or female, are intended to be covered by this disclosure. Likewise, the methods of this disclosure can be applied to any race of human, including, for example, Caucasian (white), African-American (black), Native American, Native Hawaiian, Hispanic, Latino, Asian, and European. In some embodiments of this disclosure, such characteristics may be significant. In such cases, the significant characteristic(s) (e.g., age, sex, race, etc.) will be indicated. Additionally, the term “individual” encompasses both human and non-human animals. Suitable non-human animals to which antisense oligomers of this disclosure may be administered include, but are not limited to companion animals (i.e. pets), food animals, work animals, or zoo animals. Exemplary animals include, but are not limited to, cats, dogs, horses, ferrets and other Mustelids, cattle, sheep, swine, and rodents.

Antisense oligomers of this disclosure can be administered to an individual by any suitable route of administration. Examples of such routes include, but are not limited to, oral and parenteral routes, (e.g., intravenous (IV), subcutaneous, intraperitoneal (IP), and intramuscular), inhalation (e.g., nebulization and inhalation) and transdermal delivery (e.g., topical). Any methods effective to deliver an antisense oligomer of this disclosure into the bloodstream of an individual are also contemplated in these methods. For example, transdermal delivery of antisense oligomers may be accomplished by use of a pharmaceutically acceptable carrier adapted for topical administration. Antisense oligomers can be administered in the absence of other molecules, such as proteins or lipids, or they be administered in a complex with other molecules, such as proteins or lipids. For example, the use of cationic lipids to encapsulate antisense oligomers is disclosed in U.S. Pat. Nos. 8,569,256, and 6,806,084, which are incorporated herein by reference in their entirety. Similarly, the use of peptide-linked morpholino antisense oligonucleotides is disclosed in U.S. Patent Publication No. 2015/0238627, which is incorporated herein by reference.

Because antisense oligomers of this disclosure can reduce Kit-mediated responses, such antisense oligomers can be used to treat allergic conditions. Thus, in some embodiments of the presently disclosed subject matter provided is a method of treating an allergic condition in an individual, by administering to an individual in need of such treatment an antisense oligomer of this disclosure. Allergic conditions being treated can be any condition mediated by a pathway comprising Kit. Such conditions include, but are not limited to, asthma, food allergies allergic conjunctivitis, and atopic dermatitis.

### EXAMPLES

The following EXAMPLES are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Materials and Methods Used in Examples

Mast cell culture. The HMC-1.2 human MC line was purchased from MilliporeSigma (Burlington, Mass., United States of America) and cultured according to the manufacturer's instructions. The human MC line LAD2 was obtained from Drs. Kirshenbaum and Metcalfe from the Laboratory of Allergic Diseases, NIAID, NIH, Bethesda, Md., United States of America, and cultured as described in Kirshenbaum et al., 2003. BMMCs were obtained and cultured as described in Jensen et al., 2006. Experiments on mice were carried out under a protocol approved by the Institutional Animal Care and Use Committee at North Carolina State University, Raleigh, N.C., United States of America.

Transfection of MCs with ASOs. MCs were transfected as described in Cruse et al., 2013.

Receptor expression. Receptor expression was examined by flow cytometry as described in Cruse et al., 2013.

Apoptosis and viability assays. Apoptosis was assessed with FITC-Annexin V (eBioscience, a division of Thermo Fisher Scientific Inc., Waltham, Mass., United States of America). To assess viability, cells were stained with LIVE/DEAD Green Dead Cell Stain (Invitrogen, Carlsbad, Calif., United States of America), according to the manufacturer's instructions, or propidium iodide. Flow cytometry was performed on a CytoFLEX flow cytometer (Beckman Coulter, Brea, Calif., United States of America).

Proliferation assays. Proliferation was assessed with CellTrace Far Red (Invitrogen) according to the manufacturer's instructions. Cells were also stained with LIVE/DEAD Green Dead Cell Stain. Additionally, Trypan blue was used to assess viable cell counts.

Intraperitoneal injection and peritoneal lavage. The protocol was approved by the Institutional Animal Care and Use Committee at NC State University. Peritoneal lavages were carried out after intraperitoneal injections of ASOs. Mast cells were identified as Kit and IgE receptor positive cells by flow cytometry.

Intradermal injections and skin histology. The protocol was approved by the Institutional Animal Care and Use Committee at North Carolina State University. ASOs were injected into the skin by ID injection and skin sections taken. MCs were identified and counted with toluidine blue staining and H&E was used to assess tissue morphology.

### Supplemental Materials and Methods for the Examples

Mast cell culture: The HMC-1.2 human MC line was purchased from MilliporeSigma (Burlington, Mass., United States of America) and cultured in Iscove's Modified Dulbecco's Medium (IMDM; MilliporeSigma) supplemented with 10% (vol/vol) fetal bovine serum (FBS) and penicillin (100 U/mL)/streptomycin (100 μg/mL), to according to the manufacturer's instructions. The human MC line LAD2 was obtained from Drs. Kirshenbaum and Metcalfe from the Laboratory of Allergic Diseases, NIAID, NIH and cultured, as described in Kirshenbaum et al., 2003, in StemPro-34 medium containing StemPro-34 Nutrient Supplement, L-glutamine (2 mM), penicillin (100 U/mL)/streptomycin (100 μg/mL) (Gibco; Gaithersburg, Md., United States of America) with 100 ng/mL recombinant human SCF added (R&D Systems, Minneapolis. Minn., United States of America). One-half of the medium supplemented with SCF was changed once per week. Bone marrow-derived MCs (BMMCs) were developed from bone marrow obtained from femurs of C57BL/6J mice (The Jackson Laboratory, Bar Harbor, Me., United States of America), as described in Jensen et al., 2006, and supplemented with recombinant murine 20 ng/mL of murine recombinant SCF and IL-3 (R&D Systems). Experiments on mice were carried out under a protocol approved by the Institutional Animal Care and Use Committee at North Carolina State University.

Transfection of MCs with ASOs. For transfection of MCs with ASOs, 2×10⁶ human MCs or 3×10⁶ mouse BMMCs were used for each transfection. Transfection was achieved using the Nucleofector II and Cell Line Kit V (Lonza) as described in Cruse et al., 2013. Transfection efficiency was determined using 10 μM FITC-conjugated standard control 25-mer ASO (Gene Tools LLC). Program T-030 was used for LAD2 and HMC-1.2 cells; X-001 was used for mouse BMMCs. Cell viability was monitored during experiments, and there was no evidence of cytotoxicity with ASO transfection. Standard control ASO and KitStop ESO used for experiments were unconjugated.

ASO design. KitStop ESO was designed to target exon 4 of human c-KIT (Accession No. NM_000222.2 of the GENBANK® biosequence database). A region including the splice donor site was targeted with the following sequence for human c-KIT: 5′-GAATGAAGCGATTCACTCACCTGAC-3′ (SEQ ID NO: 1). For murine c-kit, a region including the splice donor site of murine c-Kit (Accession No. NM_001122733.1 of the GENBANK® biosequence database) was targeted with the following sequence: 5′-AGGACTTAAACAGCACTCACCTGAG-3′ (SEQ ID NO: 2). All oligonucleotides were purchased from Gene Tools LLC. The unconjugated standard control ASO provided by Gene Tools LLC, does not match any known sequence in mammalian genomes and had the following sequence: 5′-CCTCTTACCTCAGTTACAATTTATA-3′ (SEQ ID NO: 3). The standard control ASO has identical chemistry to the KitStop ESO, but because it does not induce exon skipping of any known gene, the standard control ASO an ESO. For in vivo studies, morpholino ESOs linked through the terminal 3′-N to an octaguanidinium dendrimer (Vivo Morpholino) were purchased from Gene Tools, LLC, Philomath, Oreg., United States of America.

RT-PCR. LAD2, HMC-1.2, and mouse BMMCs were transfected as described above. For LAD2 and HMC-1.2 cells, total RNA was harvested after 48 hours using the RNAeasy plus mini-kit (Qiagen, Germantown, Md., United States of America) according to the manufacturer's instructions with inclusion of the QIAShredder step. For mouse BMMCs, total RNA was harvested after 24 hours following the same protocol. RT-PCR was carried out using the Thermo Fisher Scientific Verso 1-Step RT-PCR kit according to the manufacturer's instructions. The primers used were designed to amplify exon 4 and the surrounding exons and to always spanned multiple exons of c-Kit mRNA targeted by KitStop ESO. For human c-KIT mRNA, the following primers were used: Forward: 5′-GAAGCCTCTTCCCAAGGACT-3′ (SEQ ID NO: 4), Reverse: 5′-GTGTTCAGGTTTGGGGAATG-3′ (SEQ ID NO: 5). For murine c-Kit mRNA, the following primers were used: Forward: 5′-TCATCGAGTGTGATGGGAAA-3′ (SEQ ID NO: 6), Reverse: 5′-TCACAGGGGAGATGTTGATG-3′ (SEQ ID NO: 7).

Receptor expression. LAD2 and HMC-1.2 cells were transfected with standard control ASO or KitStop ESO, as described above, and incubated for 2-7 days and 24-72 hours, respectively, in complete medium. Transfected BMMCs were incubated for 48 hours in complete medium. For human cells, flow cytometry for surface and total Kit expression was performed at each time point with APC-conjugated anti-human CD117 monoclonal antibodies (clone 104D2) alongside APC-conjugated mouse IgG1, κ, isotype control antibodies (Biolegend, San Diego, Calif., United States of America). For mouse cells, flow cytometry was used to assess expression of surface FcεRIα and Kit using the following antibodies: PE-conjugated anti-mouse FcεRIα (clone MAR-1) monoclonal antibodies alongside PE-conjugated IgG isotype control (eBioscience via Thermo Fisher, Waltham, Mass., United States of America); and FITC-conjugated anti-mouse CD117 (clone 2B8) monoclonal antibodies alongside FITC-conjugated IgG2b, κ, isotype control antibodies (BD Biosciences, San Jose, Calif., United States of America).

Apoptosis and viability assays. HMC-1.2 cells (2×10⁶) were transfected with standard control ASO or KitStop ESO as described above, and incubated for 24-72 hours in complete IMDM. At each time point, cells were pelleted and analyzed for apoptosis with FITC-Annexin V (eBioscience). To assess viability, cells were stained with LIVE/DEAD Green Dead Cell Stain (Invitrogen), according to the manufacturer's instructions, or propridium iodide. Flow cytometry was performed on a CytoFLEX flow cytometer (Beckman Coulter).

Proliferation assays. Prior to transfection, HMC-1.2 cells were washed once with PBS. Cells were stained with 1 μM CellTrace Far Red (Invitrogen) at a concentration of 1×10⁶ cells/mL, according to the manufacturer's instructions. Briefly, cells were stained for 20 minutes at 37° C., followed by an addition of 5× volume of culture media for 5 minutes. Cells were pelleted, then resuspended in complete medium and incubated for 10 minutes at 37° C. to allow the CellTrace reagent to undergo acetate hydrolysis. Stained cells were transfected with standard control ASO or KitStop ESO as described above. HMC-1.2 cells were cultured at 37° C. for 24-72 hours. At each designated time point, cells were stained with LIVE/DEAD Green Dead Cell Stain, which was used to identify and gate out dead cells. Proliferating cells were identified with an additional gate set for the single-cell population. Flow cytometry was performed on a CytoFLEX flow cytometer. Additionally, Trypan blue was used to assess viable cell counts across different time points.

Intraperitoneal injection and peritoneal lavage. Four to eight week old female BALB/c mice were purchased from The Jackson Laboratory and allowed at least 1 week for acclimation prior to enrollment in the intraperitoneal injection study, which was approved by the Institutional Animal Care and Use Committee at North Carolina State University. Treatment groups were administered 50 μl of 0.5 mM KitStop Vivo morpholino ESO or control Vivo morpholino ASO via intraperitoneal injection with a 29 gauge needle at days 0 and 3 under gas anesthesia sedation to prevent trauma and blood contamination during injection. Following euthanasia by AVMA-approved guidelines on day 5, peritoneal fluid was collected via peritoneal lavage. 5 mL of ice cold PBS containing 5% w/v BSA was injected through the abdominal muscles following reflection of the skin overlying the abdomen. The abdomen was gently massaged for 30 seconds and 3-4 mL of peritoneal fluid mixed with lavage fluid was recovered by aspiration into a syringe and transferred into a conical tube on ice for transport. Total mast cell counts by surface Kit and IgE receptor expression were examined by flow cytometry of 1×10⁶ cells from the peritoneum. For RT-PCR, 5×10⁶ cells were pelleted and flash frozen on liquid nitrogen. Total RNA was harvested and RT-PCR was carried out as described above.

Intradermal injections and skin histology. Four to eight week old female BALB/c mice were purchased from The Jackson Laboratory and allowed at least 1 week for acclimation prior to enrollment in the skin injection study, which was approved by the Institutional Animal Care and Use Committee at North Carolina State University. Mice were administered 2.5 nmol the both KitStop Vivo morpholino ESO and control Vivo morpholino ASO into separate sites of the skin overlying the dorsum 1.5 cm lateral to midline via repeated injection with a 29 gauge needle at days 0, 7 and 14 under gas anesthesia sedation to allow precise injections. Following euthanasia by AVMA-approved guidelines on day 16, 1 cm×1 cm skin sections were excised, fixed for at least 48 hours in 10% neutral buffered formalin, sectioned in 5 micron steps, and stained with either toluidine blue or hematoxylin and eosin. Microscopic examination of skin sections was performed by two ACVP-board certified pathologists wherein one pathologist was blinded to treatment groups. Tallies for intact mast cells containing a nucleus within the plane of section were performed on toluidine blue stained skin sections. Tallies were collected along the entire length of two 5 micron thick tissue sections representing the opposing halves of a cross section through the previously collected 1 cm×1 cm excised formalin-fixed paraffin-embedded skin tissue.

Humanized xenograft mast cell neoplasia model. NSG mice (NOD-scid IL2R γ^{null}) from Jackson laboratories (Bar Harbor, Me., USA) (6-8 weeks) were injected subcutaneously with 1×10⁶ HMC-1.2 neoplastic MCs into the right flank. Tumor size was measured with a Mitutoyo IP65 caliper. Once tumors reached 50 mm³, mice were injected either i.v. for systemic administration, or subcutaneously near the site of the tumor every 3 days for a maximum of 14 days. For i.v. administration, 8.33 mg/kg KitStop ESO was injected, and for s.c. KitStop, 3.33 mg/kg was injected. Tumor size was measured every day and mice were euthanized when tumors reached 1.5 cm in one dimension or at day 14.

### Example 1

### Skipping of Wild-Type and Mutant c-Kit Exon 4 in ESO-Treated MCs

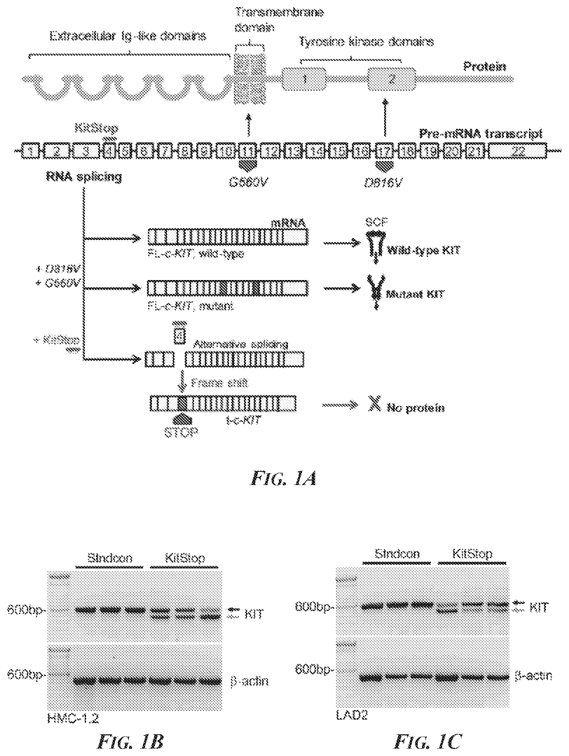

Greater than 95% transfection efficiency of ESOs in human and mouse MCs is achieved with no evidence of cytotoxicity, as determined by propidium iodide and Live/Dead staining (Cruse et al., 2016). Analyzing the sequence of human c-KIT pre-mRNA, it was predicted that ESO-induced skipping of exon 4 would introduce a frameshift into the mature mRNA open reading frame. The predicted protein translation from the mRNA contains a premature STOP codon. Thus, frameshifting with ESOs would either induce early termination of c-Kit mRNA translation, resulting in a severely truncated protein, or induce nonsense-mediated mRNA decay (NMD; FIG. 1A). Either outcome to should eliminate expression of Kit receptor. A stable 25-mer morpholino ESO was designed to target the donor splice site of exon 4 in c-Kit pre-mRNA that was termed KitStop. Since the splice site is located early in the mRNA transcript, if production of a truncated protein occurs, this protein would be severely truncated (FIG. 1A). HMC-1.2 cells exhibiting G560V and D816V mutations were tested, as were LAD2 cells, which express wild-type Kit. KitStop induced exon skipping of both mutated c-Kit (FIG. 1B) and wild-type (FIG. 1C) mRNA as indicated by RT-PCR, compared with cells transfected with an equivalent 25-mer standard control ASO.

### Example 2

### Loss of Kit Expression with c-Kit Exon Skipping

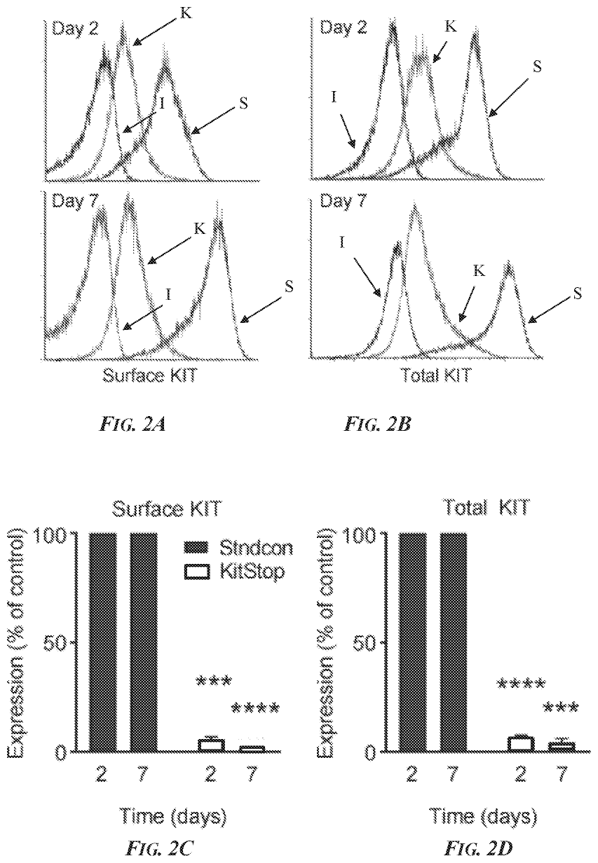

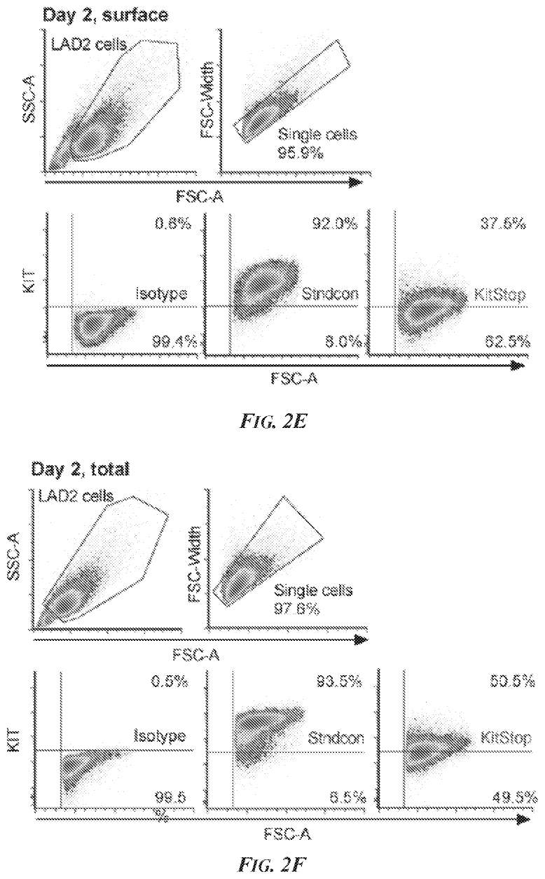

To test whether the introduction of a frameshift into either wild-type or mutant c-Kit mRNA would prevent expression of Kit protein, flow cytometry was used to measure Kit expression in LAD2 and HMC-1.2 cells (for gating strategies, see FIGS. 2E and 2F). MCs express wild-type Kit on their cell surface, but Kit is rapidly internalized following activation by its ligand, SCF. After endocytosis, Kit signals within intracellular compartments before degradation (Wiley & Burke, 2001). Accordingly, both surface (FIGS. 2A & 2C) and total Kit expression (FIGS. 2B & 2D) was measured in LAD2 cells. KitStop reduced surface Kit expression by 94.5±1.4% after 48 hours and by 99.0±0.6% after 7 days (FIG. 2C). In the same cells, total Kit expression was reduced by 93.5±0.9% after 48 hours, and by 95.6±1.8% after 7 days (FIG. 2D). These data demonstrate an almost complete loss of surface and total wild-type Kit expression in KitStop treated human MCs.

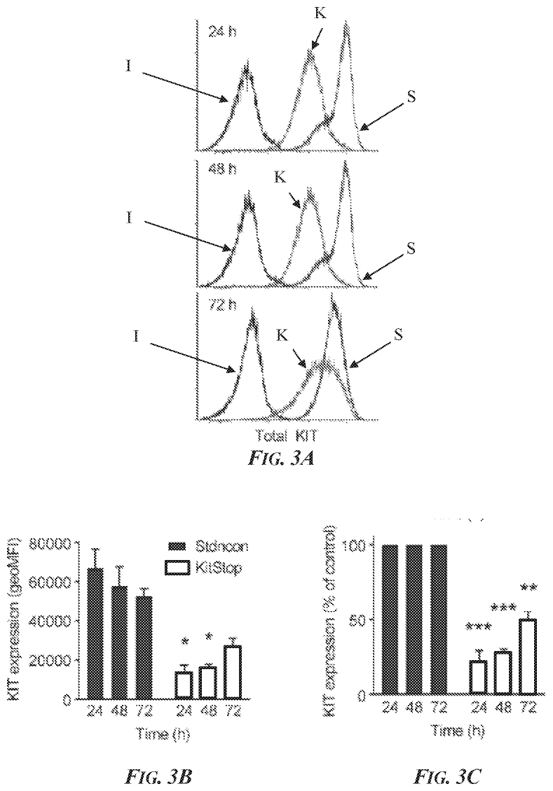

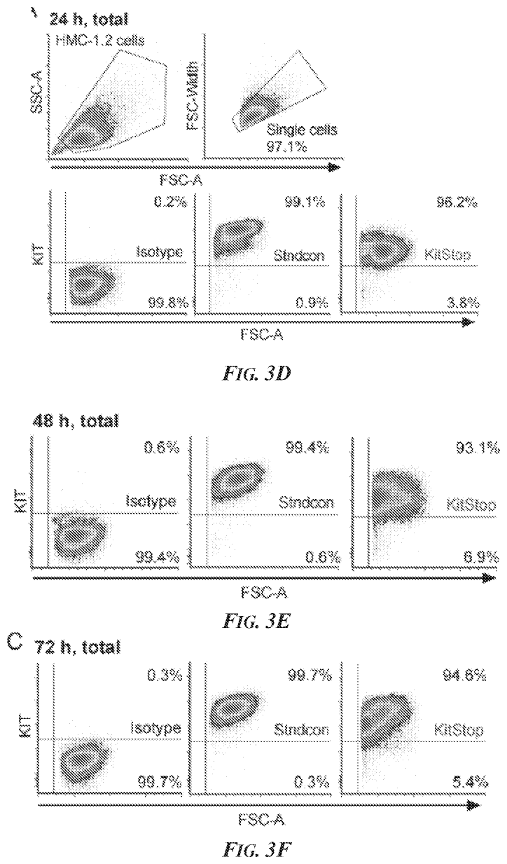

In neoplastic MCs such as the HMC-1.2 cell line, oncogenic Kit signaling occurs within intracellular compartments, independently of SCF (Obata et al., 2014). Consequently, total Kit expression was measured in HMC-1.2 cells (for gating strategies, see FIGS. 3D-3F). Since HMC-1.2 cells proliferate significantly more than LAD2 cells, KIT expression was assessed at shorter time points (FIG. 3A) and it was found that KitStop significantly reduced total mutant KIT expression (FIGS. 3B and 3C). KIT expression partially recovered over 72 hours in HMC-1.2 cells (FIGS. 3B and 3C), but not in LAD2 cells (FIG. 2D). However, the total number of viable cells in the KitStop treated HMC-1.2 cells was lower at 24 hours and reduced over the 72 hours time course compared to standard control ASO-treated cells. Since transfection efficiency is ˜95%, it is likely that a small fraction of cells that were not efficiently transfected with ESO remained after 72 hours and the efficiently transfected cells lost Kit expression and consequently diminished during the experiment.

### Example 3

### Constitutive KIT Signaling in HMC-1.2 Cells is Diminished with KitStop

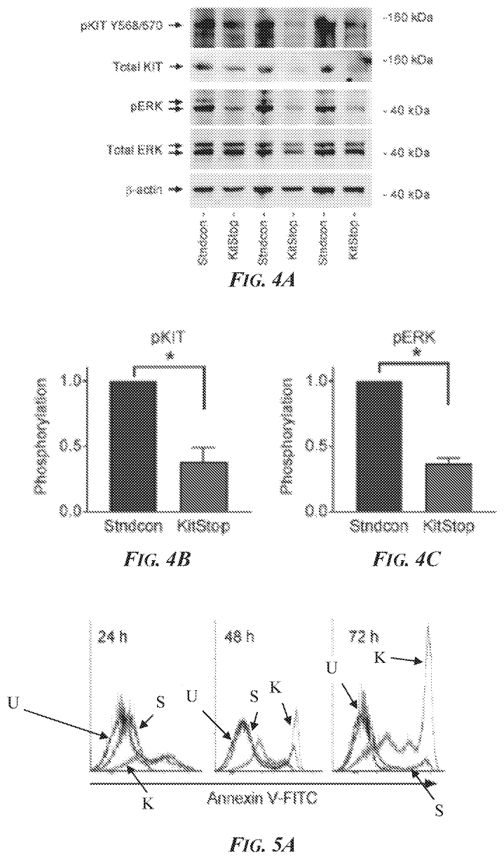

The KIT D816V mutation confers constitutive KIT signaling. As further evidence that the effects of KitStop on HMC-1.2 cell survival and proliferation were through KIT-dependent signaling, phosphorylation of KIT and the downstream kinase ERK was examined. ERK was chosen because it is a downstream kinase in the Ras-Raf-MEK-ERK pathway, which is both downstream of KIT signaling and has well established roles in cell proliferation. Both KIT and ERK were constitutively phosphorylated in HMC-1.2 cells (FIG. 4A). Transfection of the KitStop ESO reduced phosphorylation of both KIT (FIG. 4B) and ERK (FIG. 4C) by comparable amounts. Because KitStop effectively reduced functional KIT protein expression rather than inhibiting KIT phosphorylation, this reduction in phosphorylation of KIT was likely a direct result of reduced expression of the constitutively active KIT protein. However, KitStop did not affect ERK expression, compared to β-actin (FIG. 4A), thus the downstream effects on the ERK pathway were due to reduced phosphorylation, which was likely a direct result of reduced KIT protein.

### Example 4

### Frameshifting c-Kit Induced Neoplastic MC Death

In contrast to non-transformed MCs, which rapidly undergo apoptosis in the absence of exogenous supporting cytokines in vitro (Mekori et al., 1993; Yanagida et al., 1995; Asai et al., 2001), activating c-Kit mutations such as D816V enable constitutive Kit activation. In neoplastic MC diseases and cell lines such as HMC-1.2, constitutive Kit activation enables continuous SCF-independent MC survival and growth. Therefore, it was examined whether KitStop treatment can eliminate the pro-survival effects of imatinib-insensitive activating c-Kit mutations in neoplastic HMC-1.2 cells.

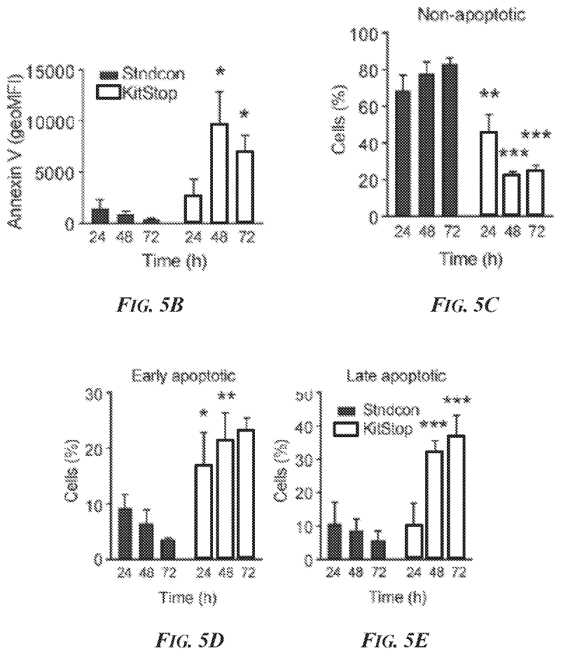

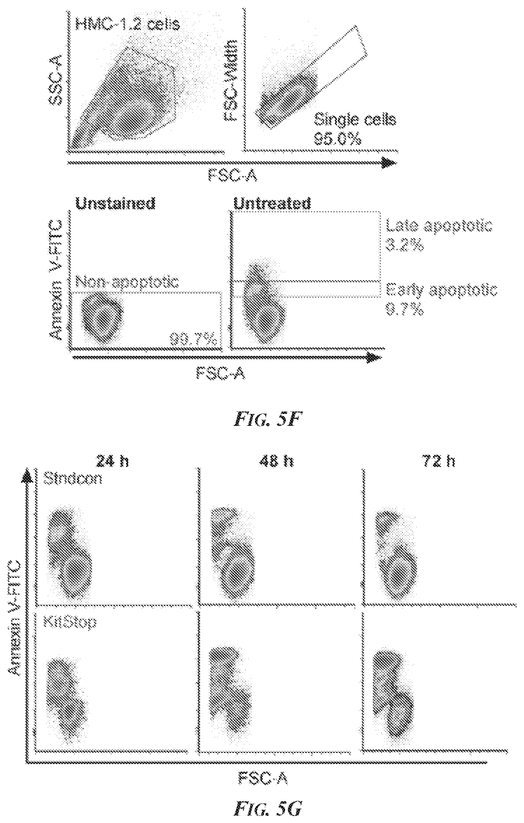

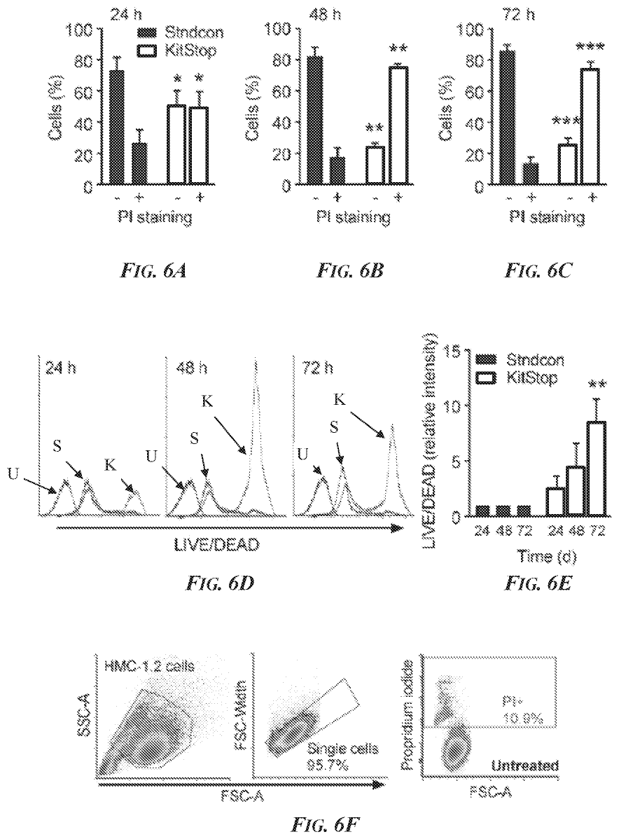

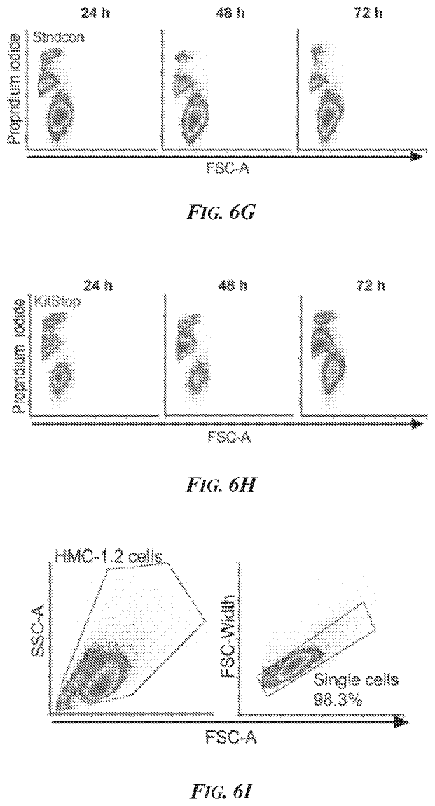

First, HMC-1.2 cell apoptosis was assessed using FITC-conjugated Annexin V (FIG. 5A; see also FIGS. 5F and 5G). In comparison to cells transfected with the standard control ASO, KitStop treated cells exhibited increased Annexin V-FITC staining over the course of 72 hours (FIGS. 5A and 5B). KitStop reduced the number of non-apoptotic cells over 72 hours (FIG. 5C) with a corresponding increase in early (FIG. 5D) and late (FIG. 5E) apoptotic cells. To examine cell viability, HMC-1.2 cells were stained with propidium iodide (PI; FIG. 6A; see also FIGS. 6F-6H). In contrast to the standard control ASO, KitStop caused increased PI staining over 72 hours (FIG. 6A-6C). A similar pattern of staining was also seen with LIVE/DEAD staining (FIG. 6D; see also 6I and 6J) with an increase in dead cells at each time point measured (FIG. 6E). Taken together, these findings demonstrate that ESO-mediated frameshifting of the c-Kit transcript rapidly induces apoptosis and cell death in neoplastic MCs.

### Example 5

### Frameshifting c-Kit Prevented Neoplastic MC Proliferation

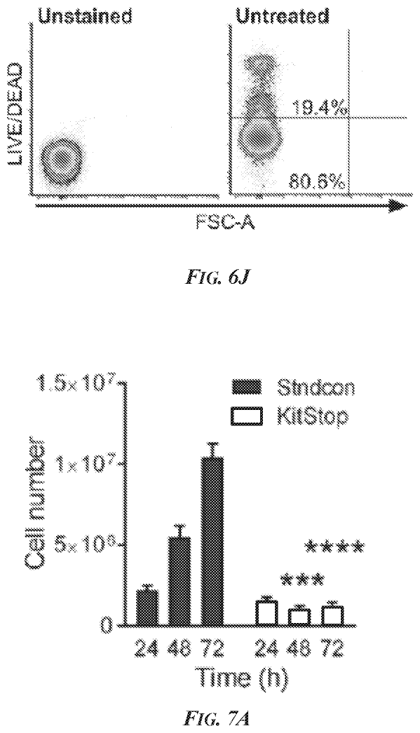

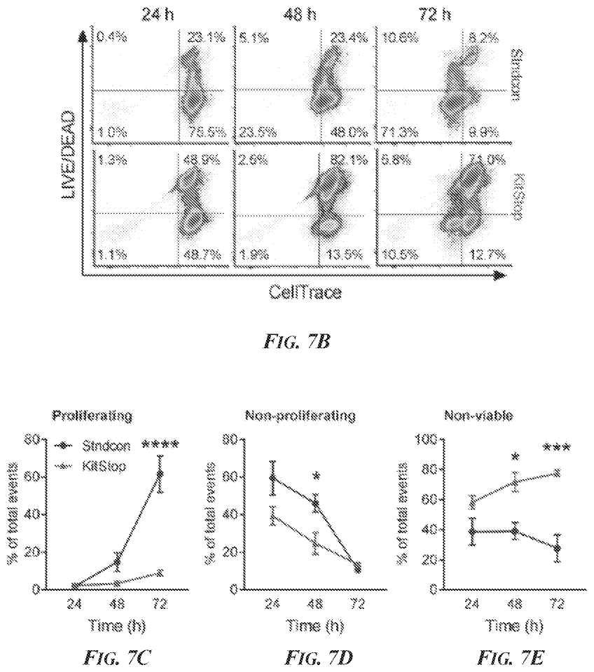

The effects of c-Kit frameshifting on neoplastic MC proliferation was also examined, given that the activating c-Kit mutations expressed by HMC-1.2 cells facilitate SCF-independent proliferation (Longley et al., 1999; Chan et al., 2013). In contrast to cells transfected with the standard control ASO, KitStop prevented HMC-1.2 growth, as demonstrated by an increase in viable cell number with trypan blue cell counts (FIG. 7A). In addition to total viable cell counts, a proliferation assay was performed using CellTrace dilution and LIVE/DEAD staining. Similarly to the cell counts, the population of viable KitStop treated cells, as determined by LIVE/DEAD staining, retained the CellTrace dye, whereas CellTrace fluorescence in standard control ASO treated cells was increasingly diluted at each time-point (FIGS. 7B and 7C). The percentage of viable non-proliferating cells reduced with time in both standard control ASO and KitStop treated cells (FIG. 7D). However, the reduction in viable non-proliferating cells with KitStop treatment was due to an increase in non-viable cells rather than cells that had proliferated (FIGS. 7B and 7E). Taken together, these findings indicate that the ESO-induced frameshifting of c-Kit markedly suppresses mutant Kit-mediated neoplastic cell growth and survival. Consequently, frameshifting oligonucleotides targeting c-Kit represent an efficient approach to reduce MC burden in vivo.

### Example 6

### In Vivo Depletion of Peritoneal MCs with KitStop ESOs

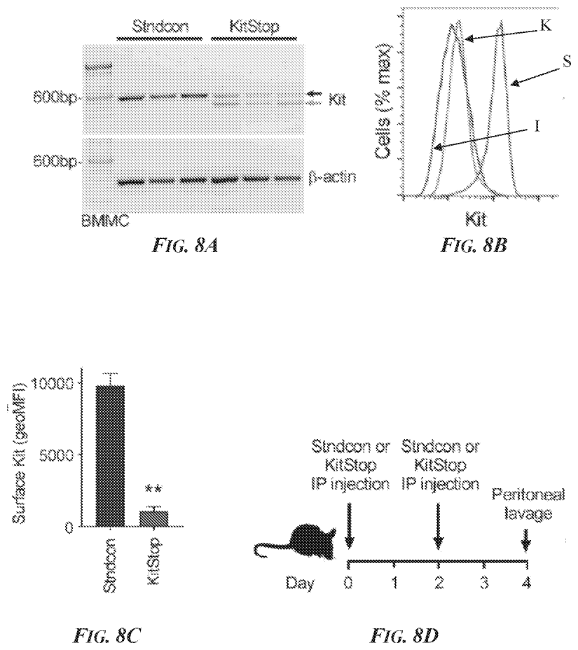

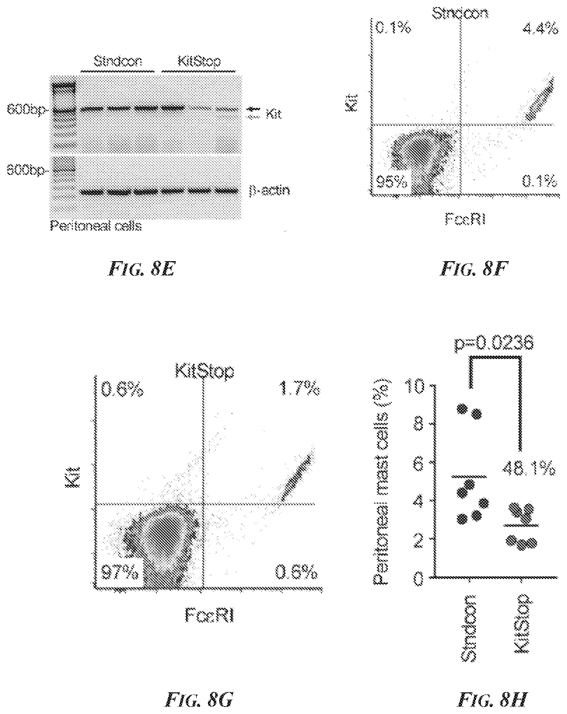

Given the success of c-Kit frameshifting oligonucleotides in neoplastic human MCs in vitro, it was sought to assess the efficacy of this approach in vivo to provide proof-of-principle for therapeutic utility. The KitStop ESO targeting mouse c-Kit was tested using mouse bone marrow-derived MCs (BMMCs). KitStop induced exon skipping of murine c-Kit mRNA, as demonstrated by RT-PCR (FIG. 8A) and marked reduction in Kit protein expression (FIGS. 8B and 8C). The efficacy of KitStop in vivo was tested using two intraperitoneal injections, followed by peritoneal lavage (FIG. 8D). Although exon to skipping of c-Kit mRNA was not always evident in peritoneal lavage cells, the level of c-Kit mRNA was markedly diminished in the majority of mice (FIG. 8E), suggesting a reduction in Kit⁺ cells in the peritoneum. Accordingly, flow cytometry of peritoneal lavage cells demonstrated that peritoneal MCs were significantly reduced by ˜50% in KitStop treated mice (FIGS. 8F-8H). Taken together, these data suggest that KitStop functions in vivo and could represent a viable approach to deplete MCs in other tissues as well.

### Example 7

### Local ESO Administration Reduced Skin MC Numbers In Vivo

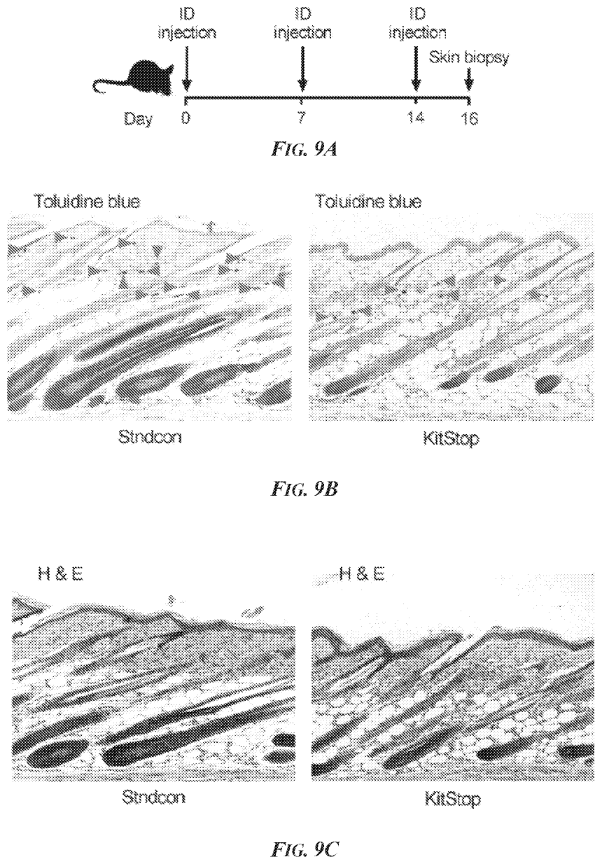

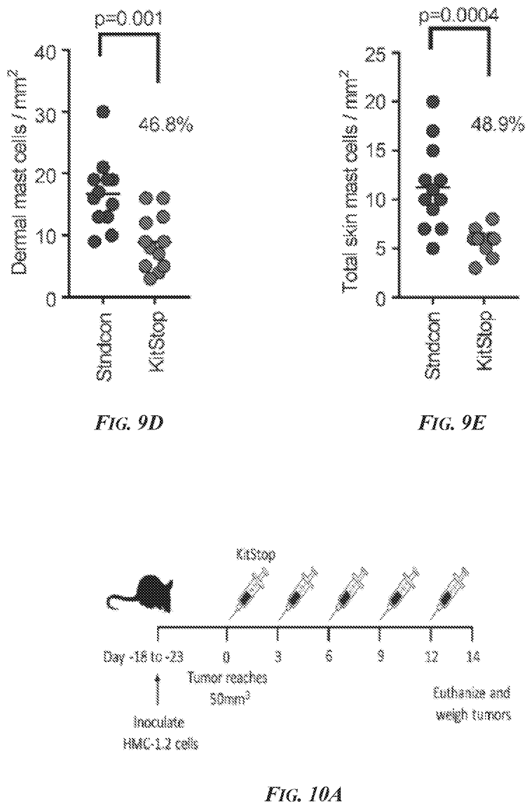

As further evidence of the therapeutic potential of KitStop in depletion of MCs in tissues, efficacy of KitStop was determined with intradermal (ID) injection into the skin of the dorsum. KitStop efficacy was assessed in skin because it has been shown that administration of ESOs into skin can effectively downregulate MC function in response to IgE signals and thus provides a model of assessing ESO activity on MCs in vivo (Cruse et al., 2016). MCs are long-lived and abundant in the skin. Therefore, the administration protocol was extended from the peritoneal studies. KitStop or standard control ASO were injected into the back skin of mice three times over a two-week period followed by assessment of skin histology (FIG. 9A). Skin sections were stained with either toluidine blue (FIG. 9B) or H&E (FIG. 9C) to identify MCs and examine histology, respectively. Two ACVP-board certified pathologists assessed MC counts and histology, one was blinded, one was non-blinded, and both pathologists drew comparable counts and conclusions. Examined tissue sections lacked overt lesions. Additionally, the KitStop treated areas of skin had nearly 50% fewer dermal (FIG. 9D) and total skin MCs (FIG. 9E) when compared to the standard control ASO-treated areas of skin taken from the same mice. These data provide further evidence that KitStop depletes mature tissue-resident MCs in vivo.

### Example 8

### Systemic Administration of KitStop ESOs Inhibits Neoplastic MC Growth In Vivo

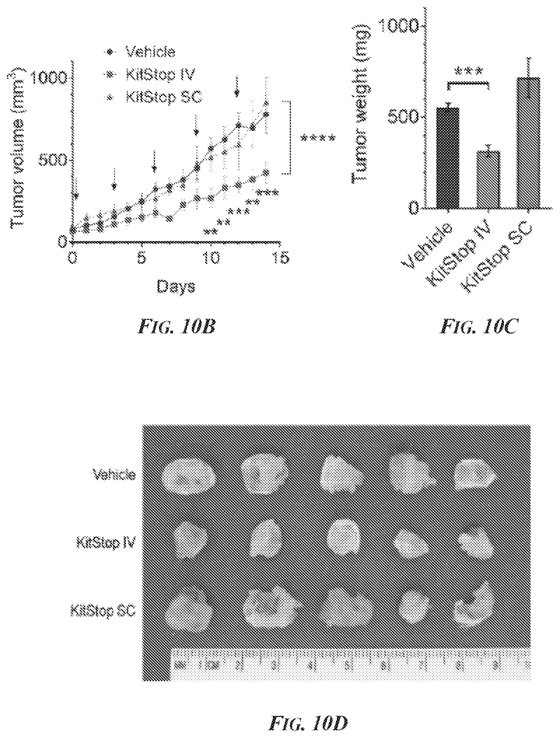

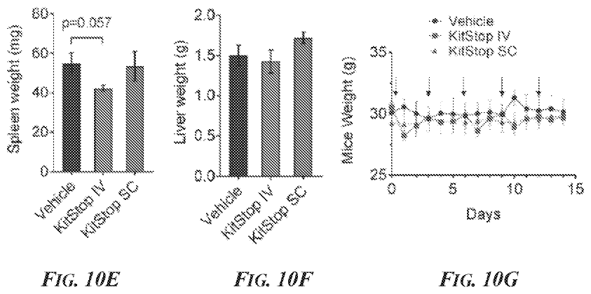

Having established that KitStop ESOs function effectively both in vitro and in vivo, KitStop was tested in a therapeutically relevant humanized in vivo model of mast cell neoplasia. NSG mice were inoculated with HMC-1.2 cells that form an aggressive tumor. Once the tumor volume reached about 50 mm³ (day 0), KitStop was administered by either intravenous (IV) or subcutaneous (SC) injection once a day every 3 days and compared to vehicle control (FIG. 10A). Using this conservative administration protocol, it was established that systemic IV administration of KitStop significantly reduced tumor growth, as measured by assessing tumor volume over time (FIG. 10B). After 14 days of treatment, mice were euthanized and the tumors were excised and weighed (FIGS. 10C and 10D). The tumors from the KitStop IV group were significantly smaller than those of both the vehicle control and KitStop SC groups. The spleens showed a trend for reduced size in the KitStop IV group (FIG. 10E), but liver weight (FIG. 10F) and mouse weight over the course of the experiment were not different (FIG. 10G).

Taken together, these data supported the conclusion that systemic (IV) administration of KitStop significantly inhibited tumor growth in an aggressive humanized mast cell neoplasia model. In addition, KitStop treatment appeared to be well tolerated and mouse weight was mostly unaffected.

### Discussion of the Examples

The role of constitutive Kit signaling in aberrant MC growth makes the receptor a target for the resulting clonal diseases, which, due to differences in patient age, organ involvement, co-morbidities and disease aggressiveness, are notoriously heterogeneous and difficult to treat (Valent et al., 2017). However, the diversity of c-Kit mutations that underlie oncogenic Kit signaling make targeting the receptor proportionally more difficult. Among these mutations, D816V, which is present in the majority of systemic mastocytosis patients, is particularly problematic. The D816V mutation confers resistance to the TM, imatinib mesylate (Gleevec) by inducing a conformational change in the tyrosine kinase domain of Kit (Antonescu et al., 2005; Theoharides et al., 2015). An emerging treatment for D816V⁺ systemic mastocytosis is the multi-kinase inhibitor, midostaurin, which exhibits high response rates. However, there is still a risk of disease progression and transformation into leukemia even after treatment with midostaurin (Gotlib et al., 2016; Valent et al., 2017). Other neoplastic disorders such as GISTs, in which somatic gain-of-function mutations in c-Kit are also common and predictive of the risk of metastasis (Heinrich et al., 2006; Ito et al., 2014), can be treated with imatinib, but similarly to mastocytosis, imatinib sensitivity can be lost over time with the acquisition of additional c-Kit mutations (Heinrich et al., 2006; Tamborini et al., 2006). Combinations of TKIs can have synergistic inhibitory effects on neoplastic MC growth and could have therapeutic benefit. However, acquired resistance to TKIs remains a risk (Gleixner et al., 2007; Gallogly et al., 2017). Consequently, given the strong association between aberrant MC proliferation and survival with activating c-Kit mutations, a desirable approach for these diseases would be capable of specifically targeting c-Kit with high efficacy, despite the many possible mutations and conformations of the receptor.

For selective targeting of c-Kit, ASO technology represents a promising approach. Also, progress with new generations of chemical modifications that improve oligonucleotide stability, bioavailability, efficacy and delivery are exciting advances in ASO technology (Juliano, 2016; Godfrey et al., 2017; McClorey & Banerjee, 2018). ASOs are versatile and, depending on their design, are capable of performing a wide range of gene-manipulating applications. For example, some applications of ASOs are to block protein translation, promote exon inclusion, or induce exon skipping of mature mRNA. In the present Examples, ESOs were used to achieve the latter, and to introduce a premature STOP codon in c-Kit mRNA. In doing so, both wild-type and mutant Kit expression was prohibited, but also Kit-dependent neoplastic MC growth and survival in vitro was also significantly inhibited. In neoplastic HMC-1.2 cells, KitStop downregulated intracellular Kit expression, which is particularly significant because the D816V mutation eliminates the need for surface expression of Kit. Instead, oncogenic Kit signaling occurs intracellularly and independently of ligand binding (Obata et al., 2014) and thus lack of mutant Kit expression at the surface negates antibody-based therapeutic approaches. In addition to the in vitro findings, it was showed that administration of KitStop reduced the number of peritoneal MCs and skin MCs in the tissues of mice in vivo. Therefore, KitStop can reduce MC burden. KitStop reduced MC numbers in the peritoneal cavity by ˜50% after four days, and a similar reduction in mature skin mast cells was evident after three treatments. The impact of KitStop on skin MC numbers is particularly important for establishing the potential efficacy of KitStop in diseases such as mastocytosis, since skin involvement is common in this disease, and because skin MCs have long life spans, and ex vivo skin MCs are resistant to growth factor and SCF withdrawal (Hazzan et al., 2017).

Two representative reasons for choosing to modify pre-mRNA splicing, rather than target c-Kit using conventional siRNA approaches, are the sheer abundance of c-Kit transcripts expressed by MCs and the inefficacy of siRNA approaches on MCs, particularly in vivo. KitStop targeting of c-Kit mRNA resulted in apparent inefficient exon skipping in human and mouse MCs when measured by RT-PCR. Inefficient exon skipping was most likely due to stoichiometry and the abundance of c-Kit mRNA transcripts compared to ESOs in the cell. Exon skipping efficiency is improved with testing different ESOs that target other splicing regulatory sites of the exon. However, even with apparent inefficient exon skipping, KitStop efficiently reduced wild type and mutant Kit protein expression with loss of proliferation and marked induction of apoptosis and cell death. KitStop may result in production of a severely truncated C-terminal protein. CRISPR/Cas systems that introduce a frameshift into transcripts result in NMD of transcripts, but NMD is often inefficient and C-terminal truncated proteins are produced that are difficult to detect (Reber et al., 2018). Thus, in contrast to conventional siRNA approaches that rely on RNase H or RNA-induced silencing complex (RISC)-mediated pathways of transcript degradation, inefficient of saturated mRNA decay with ESOs may result in production of non-functional or dominant negative proteins rather than losing efficiency of knockdown. While it is not desired to be bound by any particular theory of operation, for high copy number non-desirable transcripts, such as mutant c-Kit, this phenomenon likely plays a role in therapeutic success. Nevertheless, because ESOs are reversible manipulation of transcripts, which do not alter the genome, production of a severely truncated protein would be temporary. Therefore, the presently disclosed subject matter has advantages in certain therapeutic applications over CRISPR/Cas systems that must overcome internal regulatory processes in certain cell types in order to make the targeted changes to the gene of interest (Haapaniemi et al., 2018; Ihry et al., 2018).

In summary, the presently disclosed in vitro and in vivo data are proof-of-concept for developing ESO-induced frameshifting of c-Kit as a therapeutic treatment for MC neoplastic diseases. These experiments with wild-type mice were proof-of-concept steps towards assessing the therapeutic potential of KitStop. As has been discussed previously (Cruse et al., 2016), ESOs represent reversible therapies thereby avoiding irreversible alteration of the genome. The nonsense frameshift induced by KitStop represents a promising approach for treatment of MC neoplasia and GISTs, and represents a Kit-specific therapeutic strategy that has translational potential for clinical applications. Furthermore, c-Kit plays a role in other types of cancer, including those that are Kit-driven, such as certain melanomas, and those that acquire secondary c-Kit mutations, such as small cell lung cancer and brain tumors, in which c-Kit mutations subsequently contribute to tumor progression (reviewed in Pittoni et al., 2011). The therapeutic potential of ESOs used to alter mRNA splice variants and protein isoforms have already been highlighted in diseases such as hypercholesterolemia and cardiovascular disease (Disterer et al., 2013), breast cancer (Wan et al., 2009), and allergy (Cruse et al., 2016). Although improvements can be made towards specific delivery platforms for ESOs, the recent approval of the ESO eteplirsen for the treatment of DMD (Dowling, 2016; Syed, 2016) demonstrates increased likelihood that other ESOs will emerge as personalized and targeted therapeutics.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Antonescu et al. (2005) Clin Cancer Res 11(11): 4182-4190.
Arock et al. (2015) Leukemia 29(6): 1223-1232.
Asai et al. (2001) Immunity 14(6): 791-800.
Berger et al. (2000) Nuc Acid Res 28:2911-2914.
Besmer et al. (1986) Nature 320(6061): 415-421.
Chan et al. (2013) Clin Exp Dermatol 38(5): 538-544.
Cruse et al. (2013) Immunity 38(5): 906-917.
Cruse et al. (2014) Immunol Allergy Clin North Am 34(2): 219-237.
Cruse et al. (2016) Proc Natl Acad Sci USA 113(49): 14115-14120.
Disterer et al. (2013) Mol Ther 21(3): 602-609.
Dowling (2016) Nat Rev Neurol 12(12): 675-676.
Galli et al. (1995) Int Arch Allergy Immunol 107(1-3): 51-53.
Gallogly et al. (2017) Ther Adv Hematol 8(9): 245-261.
Giebel et al. (1992) Oncogene 7(11): 2207-2217.
Gleixner et al. (2006) Blood 107(2): 752-759.
Gleixner et al. (2007) Haematologica 92(11): 1451-1459.
Godfrey et al. (2017) EMBO Mol Med 9(5): 545-557.
Gotlib et al. (2016) N Engl J Med 374(26): 2530-2541.
Haapaniemi et al. (2018) Nat Med 24(7):927-930.
Hazzan et al. (2017) Cell Death Discov 3: 17048.
Heinrich et al. (2006) J Clin Oncol 24(29): 4764-4774.
Iemura et al. (1994) Am J Pathol 144(2): 321-328.
Ihry et al. (2018) Nat Med 24(7):939-946.
Ito et al. (2014) Int J Clin Exp Pathol 7(11): 8024-8031.
Jensen et al. (2006) Curr Protoc Immunol Chapter 3: Unit 3.23.
Jensen et al. (2008) Br J Pharmacol 154(8): 1572-1582.
Juliano (2016) Nucleic Acids Res 44(14): 6518-6548.
Kirshenbaum et al. (2003) Leuk Res 27(8): 677-682.
Longley et al. (1999) Proc Natl Acad Sci USA 96(4): 1609-1614.
Ma et al. (2002) Blood 99(5): 1741-1744.
McClorey & Banerjee (2018) Biomedicines 6(2): 10.3390/biomedicines6020051.
Mekori et al. (1993) J Immunol 151(7): 3775-3784.
Miettinen et al. (2002) Eur J Cancer 38 Suppl 5: S39-51.
Obata et al. (2014) Nat Commun 5: 5715.
Pardanani et al. (2003) Lancet 362(9383): 535-536.
Pittoni et al. (2011) Oncogene 30(7): 757-769.
Reber et al. (2018) Mol Biol Cell 29(2): 75-83.
Syed (2016) Drugs 76(17): 1699-1704.
Tamborini et al. (2006) Oncogene 25(45): 6140-6146.
Taniguchi et al. (1999) Cancer Res 59(17): 4297-4300.
Theoharides et al. (2015) N Engl J Med 373(19): 1885-1886.
Tsai M et al. (1991) Proc Natl Acad Sci USA 88(14): 6382-6386.
U.S. Pat. Nos. 6,806,084; 8,569,256; 8,765,703; 8,946,183.
U.S. Patent Publication Nos. 2015/0238627; 2015/0376615.
Valent et al. (2017) Cancer Res 77(6): 1261-1270.
Wan et al. (2009) Int J Cancer 124(4): 772-777.
Wiley & Burke (2001) Traffic 2(1): 12-18.
Wu et al. (2012) Int J Mol Med 30(1):63-68.
Yanagida et al. (1995) Blood 86(10): 3705-3714.
Yang et al. (2015) Am J Transl Res 7(9):1499-1509.

It will be understood that various details of the presently disclosed subject matter can be changed without departing from the scope of the presently disclosed subject matter. Furthermore, the foregoing description is for the purpose of illustration only, and not for the purpose of limitation.

1. An antisense oligomer comprising 10 to 50 linked nucleotides, wherein the antisense oligomer is targeted to a region of a Kit-encoding pre-mRNA, and wherein the targeted region comprises sequences involved in splicing of the Kit-encoding pre-mRNA.

2. The antisense oligomer of claim 1, wherein hybridization of the antisense oligomer to the Kit-encoding pre-mRNA alters splicing of the pre-mRNA.

3. The antisense oligomer of claim 1, wherein hybridization of the antisense oligomer to the Kit-encoding pre-mRNA reduces expression of Kit protein.

4. The antisense oligomer of claim 3, wherein the Kit protein for which expression is reduced is a wild type Kit protein or a mutant Kit protein.

5. The antisense oligomer of claim 1, wherein the targeted region comprises at least a portion of a polynucleotide sequence selected from the group consisting of an intron sequence, an exon sequence, a sequence comprising an intron/exon junction, a splice donor sequence, a slice acceptor sequence, a splice enhancer sequence, a splice branch point sequence, or a polypyrimidine tract.

6. The antisense oligomer of claim 5, wherein the polynucleotide sequence is associated with a Kit exon selected from the group consisting of Kit exons 2-20, such as but not limited to an exon 4 splice donor sequence.

7. The antisense oligomer of claim 1, wherein the Kit-encoding pre-mRNA is transcribed from a c-Kit gene.

8. The antisense oligomer of claim 1, wherein the Kit protein is selected from the group consisting of a human KIT protein, a murine Kit protein, a canine Kit protein, a feline Kit protein, and an equine Kit protein.

9. The antisense oligomer of claim 7, wherein hybridization of the antisense oligomer to the c-Kit pre-mRNA results in production of a mature c-Kit mRNA molecule that lacks at least a portion of exon 4.

10. The antisense oligomer of claim 8, wherein hybridization of the antisense oligomer to the c-Kit pre-mRNA results in production of an mRNA molecule encoding a truncated Kit protein.

11. The antisense oligomer of claim 1, wherein the 10 to 50 linked nucleotides comprises a targeting nucleic acid sequence sufficiently complementary to a target nucleic acid sequence in the Kit-encoding pre-mRNA, such that the oligonucleotide specifically hybridizes to the target sequence.

12. The antisense oligomer of claim 11, wherein hybridization of the antisense oligomer to the Kit-encoding pre-mRNA alters splicing of the pre-mRNA.

13. The antisense oligomer of claim 11, wherein hybridization of the antisense oligomer to the Kit-encoding pre-mRNA reduces expression of Kit protein.

14. The antisense oligomer of claim 13, wherein the Kit protein for which expression is reduced is a wild type Kit protein or a mutant Kit protein.

15. The antisense oligomer of claim 13, wherein the targeting sequence comprises at least 6 contiguous nucleobases fully complementary to at least 6 contiguous nucleobases in the target sequence.

16. The antisense oligomer of claim 11, wherein the targeting sequence is at least 80% complementary over its entire length to a similarly sized run of contiguous nucleobases in the target sequence.

17. The antisense oligomer of claim 11, wherein the target sequence comprises at least a portion of a polynucleotide sequence selected from the group consisting of an intron sequence, an exon sequence, a sequence comprising an intron/exon junction, a splice donor sequence, a slice acceptor sequence, a splice enhancer sequence, a splice branch point sequence, or a polypyrimidine tract.

18. The antisense oligomer of claim 17, wherein the polynucleotide sequence is associated with a Kit exon selected from the group consisting of Kit exons 2-20, such as but not limited to an exon 4 splice donor sequence.

19. The antisense oligomer of claim 11, wherein the Kit-encoding pre-mRNA is transcribed from a c-Kit gene.

20. The antisense oligomer of claim 11, wherein the Kit protein is selected from the group consisting of a human Kit protein, a murine Kit protein, a canine Kit protein, a feline Kit protein, and an equine Kit protein.

21. The antisense oligomer of claim 20, wherein the target sequence comprises at least a portion of a polynucleotide sequence selected from the group consisting of SEQ ID NOs: 18-22, optionally wherein the target sequence comprises at least a portion of a polynucleotide sequence selected from the group consisting of SEQ ID NOs: 23-60.

22. The antisense oligomer of claim 21, wherein the portion is at least 10 contiguous nucleotides.

23. The antisense oligomer of claim 21, wherein the target sequence comprises a sequence at least 90% identical to a subsequence of SEQ ID NOs: 18-22, optionally wherein the target sequence comprises a sequence at least 90% identical to one of SEQ ID NOs: 23-60.

24. The antisense oligomer of claim 21, wherein the target sequence hybridizes preferentially to a sequence selected from the group consisting of SEQ ID NOs: 1, 2, and 23-60.

25. The antisense oligomer of claim 20, wherein the targeting sequence comprises at least 10 contiguous nucleobases identical in sequence to at least 10 contiguous nucleobases in a sequence selected from the group consisting of SEQ ID NOs: 1, 2, and the reverse complement of one of SEQ ID NOs: 23-60.

26. The antisense oligomer of claim 20, wherein the targeting sequence comprises a sequence at least 80% complimentary to the reverse complement of at least a portion of a subsequence of SEQ ID NOs: 18-60.

27. The antisense oligomer of claim 20, wherein the targeting sequence is at least 80% identical over the full length of a sequence selected from the group consisting of SEQ ID NOs: 1, 2, and the reverse complement of one of SEQ ID NOs: 23-60.

28. The antisense oligomer of claim 20, wherein the targeting sequence is selected from the group consisting of SEQ ID NOs: 1, 2, and the reverse complement of one of SEQ ID NOs: 23-60.

29. The antisense oligomer of claim 2, wherein the c-Kit transcript comprises any of SEQ ID NOs: 8, 10, 12, 14, and 16, or an open reading frame present therein.

30. The antisense oligomer of claim 1, wherein the antisense oligomer is an antisense RNA molecule.

31. The antisense oligomer of claim 30, wherein the antisense RNA molecule comprises a modification selected from the group consisting of a nucleotide modification, an internucleotide modification, a sugar modification, a sugar-internucleotide linkage modification, and combinations thereof.

32. An expression vector encoding the antisense oligomer of claim 1.

33. The antisense oligomer of claim 1, wherein the antisense oligomer is a morpholino oligomer.

34. A pharmaceutical composition comprising the antisense oligomer of claim 1.

35. A method for modulating splicing of a Kit pre-RNA in a cell and/or a tissue, the method comprising contacting the cell and/or the tissue with the antisense oligomer of claim 1.

36. A method for inducing apoptosis in mast cells, the method comprising contacting the mast cell with the antisense oligomer of claim 1.

37. The method of claim 35, wherein the method is performed in an individual.

38. A method for treating a disease, disorder, or condition associated with Kit expression in an individual, the method comprising administering to the individual an antisense oligomer of claim 1.

39. The method of claim 38, wherein the disease, disorder, or condition associated with Kit expression is a cancer or mastocytosis.

40. The method of claim 39, wherein the cancer is a gastrointestinal stromal tumor or leukemia.

41. The method of claim 40, wherein the individual is an animal, optionally a mammal.

42. The method of claim 41, wherein the individual is a human, a mouse, a dog, a cat, or a horse.

43. A pharmaceutical composition comprising the expression vector of claim 32.

44. A pharmaceutical composition comprising the morpholino oligomer of claim 33.

45. A method for modulating splicing of a Kit pre-RNA in a cell and/or a tissue, the method comprising contacting the cell and/or the tissue with the expression vector of claim 32.

46. A method for modulating splicing of a Kit pre-RNA in a cell and/or a tissue, the method comprising contacting the cell and/or the tissue with the morpholino oligomer of claim 33.

47. A method for inducing apoptosis in mast cells, the method comprising contacting the mast cell with the expression vector of claim 32.

48. A method for inducing apoptosis in mast cells, the method comprising contacting the mast cell with the morpholino oligomer of claim 33.

49. A method for treating a disease, disorder, or condition associated with Kit expression in an individual, the method comprising administering to the individual the expression vector of claim 32.

50. A method for treating a disease, disorder, or condition associated with Kit expression in an individual, the method comprising administering to the individual the morpholino oligomer of claim 33.
